(19) United States
(12) Patent Application Publication
(10) Pub. No.: US 2017/0157291 A1
(43) Pub. Date: Jun. 8, 2017

(54) TISSUE TRANSPLANT COMPOSITIONS AND METHODS FOR USE

(71) Applicants: THE BRIGHAM AND WOMEN'S HOSPITAL, INC., Boston, MA (US); PARCELL LABORATORIES LLC, Westwood, MA (US)

(72) Inventors: Keith D. Crawford, Westwood, MA (US); Pamela Layton, Westwood, MA (US); John M. Garvey, Londonderry, NH (US)

(73) Assignees: THE BRIGHAM AND WOMEN'S HOSPITAL, INC., Boston, MA (US); PARCELL LABORATORIES LLC, Westwood, MA (US)

(21) Appl. No.: 15/204,774

(22) Filed: Jul. 7, 2016

Related U.S. Application Data
(63) Continuation of application No. 14/573,529, filed on Dec. 17, 2014, now abandoned.
Application No. 14/573,529 is a continuation-in-part of application No. 12/598,047, filed on Oct. 26, 2015.
Application No. 14/573,529 is a continuation of application No. 13/499,004, filed on May 30, 2012, now abandoned.
(60) Provisional application No. 60/927,596, filed on May 3, 2007.
(60) Provisional application No. 61/401,846, filed on Aug. 20, 2010.
(60) Provisional application No. 61/249,172, filed on Oct. 6, 2009.
(60) Provisional application No. 61/247,242, filed on Sep. 30, 2009.
(60) Provisional application No. 61/247,236, filed on Sep. 30, 2009.

(51) Int. Cl. A61L 27/38; A61K 45/06; A61K 35/545; A61L 27/36; A61K 35/15

(52) U.S. Cl. CPC A61L 27/3834; A61L 27/383; A61L 27/3608; A61L 27/3886; A61K 35/15; A61K 35/545; A61K 45/06; A61L 2430/02; A61L 2430/38; A61L 2430/34; A61L 2300/426

(57) ABSTRACT

Provided are transplants and methods for augmenting formation and restoration of organ and tissue, for example, bone formation, by administering autologous or allogeneic human embryonic-like adult stem cells (ELA cells). Also provided is a method for augmenting formation of tissues and organs by administering a transplant having ELA stem cells or combination of ELA stem cells.

### CROSS-REFERENCE TO RELATED APPLICATIONS

This application is a Continuation of U.S. application Ser. No. 14/573,529, which is a Continuation of U.S. application Ser. No. 13/499,004, which is a 35 U.S.C. §371 National Phase Entry Application of International Application No. PCT/US2010/050288 filed Sep. 24, 2010, which designates the U.S., and which claims benefit under 35 U.S.C. §119(e) to U.S. Provisional Application Ser. Nos. 61/247,236, filed Sep. 30, 2009, 61/247,242, filed Sep. 30, 2009, 61/249,172, filed Oct. 6, 2009, and 61/401,846, filed Aug. 20, 2010. U.S. application Ser. No. 14/573,529 is also a Continuation-in-Part of U.S. application Ser. No. 12/598,047, which is a 35 U.S.C. §371 National Phase Entry Application of PCT-International Application No. PCT/US2008/005742, filed May 5, 2008, which designates the U.S. and which claims benefit under 35 U.S.C. §119(e) to U.S. Provisional Application Ser. No. 60/927,596, filed May 3, 2007. The entire contents of each of the aforementioned patent applications are incorporated herein by this reference in their entirety.

### STATEMENT OF RIGHTS TO INVENTIONS MADE UNDER FEDERALLY SPONSORED RESEARCH

A portion of this work was supported by NIAMS grant AR050243. The government has certain rights in this invention.

### FIELD OF THE INVENTION

The present invention provides transplants, and method of making and using them, to treat tissues and organs needing repair, including bone, skin, and internal organs.

### BACKGROUND

Stem cells have long been known as a result of studies of tissue development. For example, in the development of hematopoietic tissues, it is well known that stem cells are characterized by stages of differentiation and commitment, e.g., a totipotent or pluripotent hematopoietic stem cell is capable of generating all types of blood cells and so is considered to be of early lineage, in contrast to leukopoietic and erythropoietic stem cells that are more differentiated and are restricted to generating white blood cells and red blood cells, respectively. The ultimate totipotent cell has long been recognized as the zygote, which is capable of generating all tissues of an adult organism, and early embryonic stages of development contain cells that retain pluripotency or totipotency.

A now discredited theory held that adult organisms contain only fully committed cells, specialized for each tissue; that mitotic replication would generate only similarly differentiated cells; and that only embryos contain totipotent and pluripotent cells. Recent searches for totipotent and pluripotent stem cells in adult tissues have generated positive findings. A series of cell surface markers have been used to characterize pluripotent cells, and sets of these markers can now be associated with stages of development.

Thus, genetic markers known to be associated with embryonic cells, are also found to be characteristic of adult stem cells. Many of these markers are transcription factors, including Oct-4, Nanog, Sox-2, Rex-1, GDF-3 and Stella. Among cell surface markers, CD13 encodes an aminopeptidase N and is a marker of mesenchymal stem cells; CD34 encodes sialomucin transmembrane protein (adhesion molecule) and is a hematopoietic marker; CD44 encodes hyaluronic acid-binding receptor and is a marker of mesenchymal stem cells and hematopoietic progenitor cells; CD45 encodes protein tyrosine phosphatase receptor type C and is a hematopoietic marker; CD73 encodes ecto-5′-nucleotidase and is a T and B cell marker; CD90 encodes Thy-1 and is a mononuclear stem cells marker; and CD105 encodes endoglin and is a mesenchymal stem cell and endothelial marker.

Pluripotent cells are generally tissue regenerative. It would be desirable to obtain pluripotent cells that express embryonic markers and few surface markers characteristic of particular differentiated tissues. Among cells described as adult stem cells are VSEL cells isolated from bone marrow (Ratajczak, MS et al., Stem Cell Rev 4:89-99, 2008), which have been previously reported by the investigators to be CD34 positive (Kucia, M. et al., Leukemia 19(7): 1118-27, 2005). MAPC cells isolated from bone marrow were characterized as CD13 positive and CD90 positive (Zeng et al, Stem Cells 24(11): 2355-66, 2006; Jiang et al., Nature 418(6893): 41-9, 2002). These cells of human origin were also reported to be positive for markers CD13 and CD90 (Aranguren et al., Blood 109(6): 2634-42, 2007). Muscle stem cells have been characterized as CD34 positive (Wada, M. et al., Dev 129(: 2987-95, 2002; Fukada, S. et al., Stem Cells 25(10): 2448-59, 2007). Certain adult stem cells express major histocompability antigens, for example, AFS cells isolated from amnion (De Coppi et al., Nat. Biotechnol 25(1):100-6, 2007) express WIC class I and WIC class II proteins, as well as CD44, CD73, CD90, and CD105. Cells expressing such markers are more likely to provoke rejection if transplanted into a host as these markers are immunogenic antigens. Cells expressing these markers could even cause graft versus host disease if transplanted as an allograft from a donor into an unmatched recipient.

Although embryonic stem cells are typically totipotent, the therapeutic use of such cells is controversial and potentially tumorigenic. What is needed is a transplant comprising adult stem cells of an early lineage that are pluripotent and that express few or no immunogenic determinants and few or no tissue antigen markers.

### SUMMARY

The invention generally provides a transplant that includes a population of early lineage adult (ELA) stem cells, that are pluripotent, and that express few immunogenic determinants or tissue antigen markers.

In one aspect, the invention generally provides a transplant that includes a population of early lineage adult (ELA) stem cells, such that the transplant does not substantially induce a T cell or NK cell mediated immune response in a transplant recipient. In an embodiment, the transplant is an allograft. In a related embodiment, the allograft is not WIC matched to a recipient human subject. In general, the ELA stem cell transplant is substantially free of erythrocyte cells. In various related embodiments, the transplant includes at least one component selected from plasma, buffers, cell culture medium, a preservative, an antibacterial agent, an antifungal agent, a conditioning agent, a cryogenic agent, a pharmaceutically acceptable salt, a growth factor, a vitamin, a hormone, or a therapeutic agent.

In another aspect, the transplant includes a carrier having a matrix that is any one or more of a gel, a connective tissue graft that is substantially depleted of living cells, a polymer scaffold, calcium triphosphate, demineralized bone, collagen, and cellulose, wherein the matrix conforms substantially to its insertion site and provides a structurally stable, three dimensional surface for retaining the transplant and supporting ingrowth of ELA stem cells into the matrix at the insertion site, such that the insertion site is the site of delivery of the transplant into the recipient subject The transplant promotes ingrowth of ELA stem cells and induces tissue formation, the tissue being ectoderm, mesoderm or endoderm, in the recipient. The tissue formation resulting from the transplant includes any or a combination of: connective tissue, bone, dermal tissue, neuronal tissue, endothelial tissue, muscle, cardiac muscle, dentin, ocular tissue and organ tissue.

In another embodiment, the transplant is cryogenically frozen. In an alternative embodiment, the transplant is freshly prepared. Without limitation, the ELA stem cell numbers in the transplant include a range of from about 5000 ELA stem cells to about 5×10⁶ ELA stem cells (e.g., 1×10³, 1×10⁴, 1×10⁵, 1×10⁶), although numbers outside this range are also envisioned as being suitable for the transplant.

An aspect of the invention provides a method of preparing a transplant that includes:

obtaining a volume of a first donation sample and a volume of a second donation sample from a human donor not exhibiting disease symptoms contraindicating the donor's suitability for tissue donation;

testing the first donation sample for detectible pathogenic microorganisms and disease markers;

processing the volume of second donation sample to obtain an enriched cellular fraction, the enriched cellular fraction comprising a population of immunomodulatory cells including early lineage adult (ELA) stem cells; and,

resuspending the cellular fraction in a volume of suitable transplant medium or preservation medium, thereby obtaining the transplant.

In various related embodiments, the first donation sample and the second donation sample are any one or more of: blood, bone marrow, serum, lymph, semen, urine, tears, synovial fluid, cerebrospinal fluid, milk, and tears. In general, testing the first donation sample includes at least one of physical exam of the donor, and a medical quiz to be filled out by the donor or the donor's physician, as well as laboratory assays on the first donation sample. In general, processing the second donation sample includes a step of centrifuging the sample to obtain the cellular fraction as a pellet. The centrifuging is optimized for speed of the centrifuge (g force or rpm) and time of centrifugation, by methods well known in the art of cell biology to obtain as many cells as possible without compromising the viability of the cells.

The detectible pathogenic microorganisms include, without limitation, viruses, bacteria, protozoa and fungi; for example, the viruses include a plurality selected from: Hepatitis B virus (HPB); Hepatitis C virus (HPC); HIV-1; HIV-2; HTLV-1; HTLV-2; Vaccinia; Varicella zoster; West Nile virus (WNV); HSV type 1, HSV type 2; and poliomyelitis; the bacteria include a plurality selected from: Staphylococcus aureus; Mycobacterium tuberculosis; and Neisseria gonorrhoeae; the fungi include at least one selected from Candida albicans; Cryptococcus neoformans; Aspergillus fumigatus; and Histoplasma capsulatum; and the protozoa include a plurality selected from: Leishmania donovani; Trypanosoma cruzi; Plasmodium falciparum; Plasmodium vivax; Plasmodium ovale; Plasmodium malariae; Babesia divergens; Babesia microti; and Babesia bovis, however, the members of this list should be adjusted by the practitioner to be suitable for the particular patient population and particular application of the transplant.

Similarly, at least one of the disease symptoms and the disease markers includes: active genital herpes; clinically active gonorrhea; systemic mycosis; Leishmaniasis; malaria; sepsis; transmissible spongiform encephalopathy (TSE); clinically significant metabolic bone disease; polyarteritis nodosa; rheumatoid arthritis; sarcoidosis; systemic lupus erythematosus; tuberculosis; Alzheimer's disease; cancer antigens; ankylosing spondylitis; antiphospholipid syndrome; autoimmune hemolytic anemia; autoimmune lymphoproliferative syndrome; autoimmune thrombocytopenic purpura; autoimmune vasculitis; Chagas disease; cold agglutinin disease; Guillain-Barre syndrome; infection with methicillin-resistant Staphylococcus aureus (MRSA); and mixed connective tissue disease.

An aspect of the invention herein provides a method of treating a subject in need of an allograft transplant, the method including:

identifying a subject having a tissue in need of repair; and

contacting the tissue of the subject with a transplant that includes ELA stem cells, such that the transplant induces growth of new tissue in the subject and does not induce an immune response in the subject against the new tissue or the transplant, and wherein the growth of new tissue in the subject thereby treats the subject. For example, the transplant used in an embodiment of the method is an autologous transplant of the subject's own ELA stem cells. Alternatively, the transplant is allogeneic to the subject, for example, the transplant is prepared from a donation sample of a human individual unrelated to the recipient. Further, the ELA stem cells are not MHC matched to the recipient subject recipient subject prior to the transplant. Alternatively, the transplant is xenogeneic to the subject, for example, the donor is a non-human mammal, including without limitation, bovine, porcine, canine, equine, and the like.

In various embodiments of the method, contacting the tissue with the transplant includes at least one route of delivering the transplant by injecting, infusing, or surgically emplacing the transplant. For example, the tissue to be contacted includes, but is not limited to, cardiac, vascular, epithelial, endothelial, dermal, corneal, retinal, dental, connective, neuronal, facial, cranial, soft tissue including cartilage and collagen, liver, kidney, spine, central nervous system, such as spine and brain, peripheral nerve, vocal cords, bone, bone marrow, and joint tissue, including articular joints. This list is merely exemplary and should not be construed as limiting. Further, the subject in need of a transplant is afflicted with a condition selected from the group consisting of: organ deterioration or failure; a cancer; a bone defect, a spinal defect requiring fusion of vertebrae, a soft tissue defect, a wound, a burn, an autoimmune disease, a demyclinating disease, myasthenia gravis; Guillain-Barre syndrome, systemic lupus erythematosis, uveitis, autoimmune oophoritis; chronic immune thrombocytopenic purpura, colitis, diabetes, Grave's disease, psoriasis, pemphigus vulgaris, rheumatoid arthritis, an inflammatory condition and an infection.

In various embodiments, the method further includes, prior to contacting the subject, exposing the transplant to one or more bioactive factors that induces or accelerates differentiation of ELA stem cells in the transplant, where the factors are any one or more of growth factors, cytokines and chemokines: bone morphogeneic proteins BMP-2, BMP-3, BMP-4, BMP-6, and BMP-7; platelet-derived growth factor (PDGF), epidermal growth factor (EGF), basic fibroblast growth factor (bFGF), interleukins such as IL-3, IL-4 and IL-1, insulin-like growth factor-1 (IGF-1), leukemia inhibitory factor (LIF), vascular endothelial growth factor (VEGF), and erythropoietin (EPO); GDF-5; transforming growth factor 13-3 (TGF-133), granulocyte colony stimulatory factor G-CSF, granulocyte-macrophage colony stimulatory factor GM-CSF, Flt-3 ligand, stem cell factor (SCF), IL-3 receptor agonists such as Daniplestim; thrombopoietin agonists; chimeric cytokines such as leridistim and progenipoietin-1, peg-filgrastim, and SDF-1 antagonists such as AMD 3100; and from the group of chemotherapeutic agents: cyclophosphamide, iphosphamide, carboplatin, etoposide (ICE), etoposide, methylprednisolone, ara-c and cisplatin.

In various related embodiments of the method, the transplant includes ELA stem cells that are culture expanded and optionally frozen. In alternative embodiments, the transplant contains cells that are freshly prepared, or contains a mixture of freshly prepared, freshly prepared and freshly expanded, and/or culture expanded and frozen cells. In related embodiments, the method includes cultured cells that are genetically modified cells.

In various embodiments of the method, the subject is afflicted with an autoimmune disease or an inflammatory condition, and the method further includes analyzing remediation by measuring a decrease or a remittance of symptoms. Alternatively or in addition, the subject is afflicted with an organ failure, a wound or a wasting syndrome, and the method further includes, respectively, analyzing remediation by measuring regeneration of organ tissue, analyzing remediation by measuring wound healing, or analyzing remediation by measuring weight gain, respectively.

An aspect of the invention provides a kit that includes a transplant, the transplant further including a sterile, buffered, isotonic preparation having a population of immunomodulatory cells and tissue progenitor ELA stem cells, the kit further including suitable aseptic packaging of the transplant and instructive indications for use of the transplant. For example, the container includes a device for delivering the transplant which is cryogenically preserved within the device. For example, the device is at least one of: a hypodermic injector; an infusion bag; and a sponge for surgical insertion at a localized target organ or tissue.

The kit in various embodiments includes cryogenically preserved cells, for example, which prior to preservation are cultured in the presence of one or more bioactive factors, for example a bone morphogenetic factor for example, any one or more of bone morphogeneic proteins BMP-2, BMP-3, BMP-4, BMP-6, and BMP-7; platelet-derived growth factor (PDGF), epidermal growth factor (EGF), basic fibroblast growth factor (bFGF), interleukins such as IL-3, IL-4 and IL-1, insulin-like growth factor-1 (IGF-1), leukemia inhibitory factor (LIF), vascular endothelial growth factor (VEGF), and erythropoietin (EPO); GDF-5; transforming growth factor β-3 (TGF-β3), granulocyte colony stimulatory factor G-CSF, granulocyte-macrophage colony stimulatory factor GM-CSF, Flt-3 ligand, stem cell factor (SCF), IL-3 receptor agonists, such as Daniplestim; thrombopoietin agonists; chimeric cytokines such as leridistim and progenipoietin-1, peg-filgrastim, and SDF-1 antagonists such as AMD 3100; and from the group of chemotherapeutic agents: cyclophosphamide, iphosphamide, carboplatin, etoposide (ICE), etoposide, methylprednisolone, ara-c and cisplatin.

In various alternative embodiments, the kit contains allogeneic or autologous cells that are any one or more of osteogenic cells, osteocytes, epithelial cells including epidermal, dermal, endothelial, chondrogenic cells, chondrocytes, chondrogenic cells, hematopoietic cells, platelets, adipogenic cells, adipocytes, neurogenic cells, astrocytes, myogenic cells and myocytes, hepatogenic cells and hepatocytes, renal cells, pancreatic cells including islet cells and beta cells, and immune cells, including B cells and T cells.

An aspect of the invention provides a method of treating a subject in need of an allograft transplant, the method including:

identifying a subject having a cutaneous tissue defect in a treatment site;

engrafting to the tissue defect site of the subject an ELA stem cell transplant comprising a population of ELA stem cells and immune cells; and,

occluding the engrafted tissue defect site with at least one of: composite allografts, biosynthetic dressings, acellular dermal allografts, autografts or cultured skin substitutes, and skin allografts or xenografts, where the transplant induces growth of new tissue including one or more of: hypodermal, dermal, and epidermal tissues of skin, connective tissue, sebaceous, vascular endothelial, cardiac muscle and neural tissues at the site, and does not induce an immune response in the subject against the transplant or resultant tissue, and where the growth of tissue in the subject thereby treats the subject.

An embodiment of the method further includes administering a vacuum to the occluded transplant.

### BRIEF DESCRIPTION OF THE FIGURES

FIG. 1 is a set of micrographs (phase contrast photomicrographs) of human ELA stem cell cultures at various stages of development. An ELA stem cell colony/embryoid body at days 0, 3, 6, and 9 of primary culture was observed composed of uniformly spindle-shaped cells. These spindle-shaped cells continue to populate the tissue culture surface until full confluence is established at day 9 of culture.

FIGS. 2A-2C are a set of photomicrographs of human ELA stem cells that had differentiated into various mesodermal tissues. ELA stem cells from the same donor were cultured for 21 days in each of adipogenic medium (FIG. 2A), chondrogenic medium (FIG. 2B) and osteogenic medium (FIG. 2C). Each of the photomicrographs shows histological features characteristic of differentiated tissues.

FIGS. 3A and 3B are a set of photographs of an ELA stem cell-loaded demineralized bone matrix implant. ELA stem cells were loaded into demineralized bone matrix and cultured 21 days in control basal medium (FIG. 3A) lacking osteogenic factors, and in osteogenic medium (FIG. 3B). Following culture, a high level of bone formation was observed as a black color in FIG. 3B, following use of stain specific for alkaline phosphatase (APase).

FIG. 4 is a bar graph showing suppression by the ELA stem cells of pan-activated T cells. T cells treated with anti-CD3 and CD28 were observed to have maximally proliferated upon exposure to these antibodies. ELA cells (bars on left, darker shade) and Mesenchymal stem cells (MSCs; bars on right, lighter shade) suppressed anti-CD3 induced proliferation and anti-CD28 induced proliferation. The graph shows that efficiency of T cell suppression was observed to decrease with an increasing concentration of the ELA cells, with the ratio of NK effector cells: K562 target cells shown on the abscissa, and percent T cell suppression shown on the ordinate. At the highest concentration of ELA stem cells (ratio 1:2 to T cells), no augmentation in T cell proliferation was noted. In contrast, MSCs augment T cell proliferation at high MSC concentrations.

FIG. 5 is a line graph showing that ELA stem cells actively suppress natural killer (NK) cell cytotoxicity. NK cells or a mixture of NK and ELA cells were cultured with target cells K562 (an immortalized human myelogenous leukemic cell line), and induction of cytotoxicity, on the ordinate, of the K562 cell was measured as a function of the ratio of effector: target cells on the abscissa. Killing by NK cells (squares) was observed to be dose dependent and decreased with increasing ratios of NK cells to the target. In contrast, NK cells that were cultured in the presence of the ELA cells (circles) and then transferred to K562 cultures were observed to have significantly decreased ability to kill the target cells. The ELA stem cells were further observed to suppress the NK cell cytotoxicity in a dose dependent manner.

FIGS. 6A-6C show QPCR analysis of ELA cells cultured in the presence of adipogenic (FIG. 6A), chondrogenic (FIG. 6B), or osteogenic media (FIG. 6C). The height of the bars shown on the ordinate reflects the number of PCR cycles used to observe expression. The dotted line in each panel represents the number of PCR cycles used to detect expression of a housekeeping gene (22 cycles), so that bars of lesser height indicate greater extent of expression than that of housekeeping genes. The data show that expression of genes in ELA cells specific to each of adipose tissue, bone and cartilage was specifically upregulated by culture of these cells in respective inducing media.

FIGS. 7A and 7B provide a set of photomicrographs showing that genetically modified

ELA cells produce green fluorescent protein. Freshly prepared ELA cells were treated with a Lentivirus vector carrying a gene encoding a GFP reporter gene that was engineered specifically to be activated by the embryonic form of OCT4. FIG. 7A is a phase contrast view of the cells. FIG. 7B is a black and white version of a photograph that in color shows green fluorescence associated with locations of the cells.

FIG. 8 is a line graph showing osteocalcin concentration on the ordinate (ng/ml) as a function of time of culture of ELA cells cultured on ProFuse® matrix (commercially available from Alphatec Spine, Inc., Carlsbad, Calif.) seeded at three different amounts of cells, 15,000 (shown as Pro15, diamonds), 30,000 (Pro30, circles) and 60,000 (Pro60, triangles), compared to that of Osteocel®, commercially available from Ace Surgical Supply Co., Brockton, Mass. Osteocalcin was measured after each of one, two, three and six weeks of culture. Data for Osteocel® was extrapolated beyond six weeks.

FIGS. 9A-9C are a set of photographs of ELA cells seeded on a matrix commercially available from Etex Corp. (Cambridge, Mass.). The matrices were seeded with cells and cultured (FIG. 9A) then photographed before (FIG. 9B) and after staining for AP (FIG. 9C).

FIGS. 10A-10F provide a set of x-ray photographs taken at two weeks and six weeks after implantation of cells and a Profuse matrix. FIGS. 10A and 10B are x-ray photographs of a rat receiving an implant of Profuse with human bone marrow aspirate (BMA). FIG. 10A was taken at two weeks after implantation, and FIG. 10B at six weeks after implantation. FIGS. 10C and 10D show an x-ray of rats that received an implantation of CD105 MSC cells. FIGS. 10E and 10F show x-ray photographs of a rat with an implant of human ELA transplant.

FIG. 11 is a bar graph showing the fluoroscopic scores from subjects shown in FIGS. 10A-10F, at each of 2, 4 and 6 weeks.

FIG. 12 is a bar graph showing results from implants made with bone chips rather than Profuse and cells from bone marrow aspirate (BMA), MSC cells, and a human ELA transplant.

### DETAILED DESCRIPTION

Isolation and purification of adult stem cells are described, for example, in U.S. utility application Ser. No. 12/598,047, filed Oct. 29, 2009, international application PCT/US2008/005742, filed Monday May 5, 2008, U.S. provisional application Ser. No. 60/927,596, filed May 3, 2007, U.S. Provisional Application Nos.: 61/247,236 and 61/247,242, both filed Sep. 30, 2009, Ser. No. 61/249,172 filed Oct. 6, 2009, and Ser. No. 61/501,846 filed Aug. 20, 2010, each of the which is hereby incorporated by reference herein in its entirety). Such cells include cells that are capable of proliferating and differentiating into ectoderm, mesoderm, and endoderm, and express Oct-4, KFL-4, Nanog, Sox-2, Rex-1, GDF-3 and/or Stella, but do not detectibly express CD13, CD45, CD90, CD34, and further do not detectibly express MHC class I, MHC class II, CD44, CD105, CD49a, CD73, CD66A, CSCR4 or an SSEA. These cells are referred to herein as early lineage adult (“ELA”) stem cells.

The present invention is directed to a transplant composition having a population of ELA stem cells. In accordance with an aspect of the present invention, a mammal is treated with ELA stem cells wherein the ELA stem cells are obtained from a mammal other than the mammal being treated, i.e. the ELA stem cells are not autologous. In accordance with an aspect of the present invention, a mammal is treated with ELA stem cells, wherein the ELA stem cells are obtained from that mammal being treated, i.e. the ELA stem cells are autologous.

Repair of large segmental defects in diaphyseal bone is a significant problem faced by orthopedic surgeons. Although such bone loss may occur as the result of acute injury, these massive defects commonly present secondary to congenital malformations, benign and malignant tumors, osseous infection, and fracture non-union. The use of fresh autologous bone graft material has been viewed as the historical standard of treatment but is associated with substantial morbidity including infection, malformation, pain, and loss of function (Kahn et al. 1995 Clin. Orthop. Rel. Res. 313:69-75). The complications resulting from graft harvest, combined with its limited supply, require the development of alternative strategies for the repair of clinically significant bone defects. The primary approach to this problem has focused on the development of effective bone implant materials.

Classes of bone implants may be categorized as osteoconductive, osteoinductive, or directly osteogenic. Allograft bone is probably the best known type of osteoconductive implant. Although widely used for many years, the risk of disease transmission, host rejection, and lack of osteoinduction compromise its desirability (1988, JAMA 260:2487-2488). Synthetic osteoconductive implants include, titanium fibermetals and ceramics composed of hydroxyapatite and/or tricalcium phosphate or bioactive tricalcium phosphate. The favorably porous nature of these implants facilitates bony ingrowth, but their lack of osteoinductive potential limits their utility. A variety of osteoinductive compounds has also been studied, including demineralized bone matrix, which is known to contain bone morphogenic proteins (BMP). Since discovery of BMP, others have characterized, cloned, expressed, and implanted purified or recombinant BMPs in orthotopic sites for the repair of large bone defects (Gerhart, et al.1993 Clin. Orthop. Rel. Res. 293:317-326, Stevenson et al. 1994 J. Bone Joint Surg. 76(11):1676-1687, Wozney et al. 1988 Science. 242:1528-1534). The success of this approach has hinged on the presence of nearby cells capable of responding to the inductive signal provided by the BMP (Lane et al. 1994 In First International Conference on Bone Morphogenic Proteins, Baltimore, Md., Jun. 8-11, abstract). These localized progenitors undergo osteogenic differentiation and have been considered responsible for synthesizing new bone at the surgical site. Nearby cells include ELA stem cells (Crawford, Keith, PCT/US2008/005742) that differentiate into the osteogenic lineage to an extent sufficient to generate bone formation.

An alternative to the osteoinductive approach is the implantation of living cells that are directly osteogenic. Since bone marrow has been shown to contain a population of cells possessing osteogenic potential, experimental therapies have been devised based on the implantation of fresh autologous or syngeneic marrow at sites in need of skeletal repair (Grundel et al. 1991 Clin. Orthop. Rel. Res. 266:244-258, Werntz et al. 1996 J. Orthop. Res. 14:85-93, Wolff et al. 1994 J. Orthop. Res. 12:439-446). Though sound in principle, the practicality of obtaining sufficient bone marrow with the requisite number of osteoprogenitor cells is limiting.

What is needed is a transplant having a population of cells having osteogenic properties that can be obtained in large concentrations. Preferably, such cells can be combined with a natural or synthetic matrix, alone or with other cells from the body or with bioactive factors that will augment the formation of bone in the body.

More particularly, in accordance with an aspect of the invention, a patient is treated with an allogeneic human ELA stem cell transplant. The ELA stem cells have been found to be immunologically neutral and therefore can be used as described herein without inducing an adverse immune response in the recipient of the transplant. In addition, applicants have found that the donor of the ELA stem cells need not be “matched” to the recipient.

In accordance with the present invention, it has been discovered that ELA stem cells are “invisible” to the immune system. Normally, co-culturing cells from different individuals (allogeneic cells) results in T cell proliferation, manifested as a mixed lymphocyte reaction (MLR). However, when human ELA stem cells are contacted with allogeneic T lymphocytes, in vitro, they do not generate an immune response by the T cells, i.e., the T cells do not proliferate, indicating that T cells are not responsive to allogeneic ELA stem cells. It has also been discovered that ELA stem cells actively reduce the allogeneic T cell response in mixed lymphocyte reactions in a dose dependent manner. Similar observations are made regarding the ability of the ELA stem cells to attenuate NK cell killing. It has further been discovered that ELA stem cells from different donors do not exhibit specificity of reduced response with regard to MEW type. Thus, ELA stem cells need not be MEW matched to a target cell population in the mixed lymphocyte reaction in order to reduce NK cell mediated cytotoxicity and the proliferative response of alloreactive T cells to an ELA stem cell transplant.

Transplants provided herein include autologous or allogeneic cells. The cells may be freshly prepared or cryopreserved, unexpanded, expanded, or master cell bank generated human derived ELA stem cells that have been shown to generate bone in vitro and are here provided as transplants to regenerate bone in vivo. Autologous or allogeneic ELA stem cells are harvested from donor biological samples, from either the recipient in the case of autologous, or from another human donor in the case of allogeneic, or from a mammal of another species (xenogeneic), primarily from bodily fluids, such as synovial fluid or peripheral blood, or from tissues, such as bone marrow. Transplants herein provide an alternative to autogenous bone grafting with its attendant invasive pretransplant surgery, and will be particularly useful in clinical settings such as ageing and osteoporosis, where there is a need to enhance bone regeneration in the spine and other bones. The transplants herein are useful also in dental applications where there is a need to regenerate bone.

Transplants according to the present invention that contain autologous or allogeneic adult stem cells are especially useful for facilitating repair, reconstruction and/or regeneration of a tissue defects. Patent applications U.S. utility application Ser. No. 12/598,047 filed Oct. 29, 2009, international application PCT/US2008/005742 filed Monday May 5, 2008, U.S. provisional application Ser. No. 60/927,596 filed May 3, 2007, U.S. provisional applications serial numbers 61/247,236 and 61/247,242, both filed Sep. 30, 2009, Ser. No. 61/249,172 filed Oct. 6, 2009, and Ser. No. 61/501,846 filed Aug. 20, 2010, each of which is hereby incorporated by reference herein in its entirety, describe isolation and purification of an early lineage adult (ELA) stem cell population, e.g., through centrifugation of synovial fluid, and the application of these ELA stem cells as an osteogenic agent, whereby either autologous or allogeneic ELA stem cells are employed in various methods and products for treating skeletal and other connective tissue disorders.

The present invention relates to transplants that utilize allogeneic ELA stem cells. The allogeneic ELA stem cells suppress activated T cells and NK cell mediated cytotoxicity in the transplant recipient. Thus, the allograft ELA transplants downregulate host immune based rejection of the transplant. Accordingly, by downregulating the host immune response, the present invention provides for enhanced transplant cellular grafting and increased transplant cell proliferation in the recipient allogeneic host.

The tissue grafts are used for a variety of medical transplant procedures. For example, they are suitable to transplant in order to enhance hematopoietic cell production in a recipient needing blood cells; enhance tooth pulp production and resultant bone, tooth and nerve formation in connection with dental procedures, enhance fibroblast production in skin grafts; enhance soft tissue and bone formation in orthopedic procedures; for treatment of connective tissue disorders, for example the tissues of the body that support the specialized elements, and include bone, cartilage, ligament, tendon, stroma, muscle and adipose tissue; to enhance nerve growth and regeneration in patients in need thereof and to reduce scarring and repopulate connective tissues in cosmetic procedures. ELA stem cells are pluripotent, and have the potential to differentiate into any of the three germ layers: endoderm (e.g., the interior stomach lining, gastrointestinal tract, the lungs), mesoderm (e.g., the muscle, bone, blood, urogenital tissues), or ectoderm (e.g., the epidermal tissues and nervous system tissues). Pluripotent stem cells can give rise to any fetal or adult cell type. Thus, ELA stem cells can be utilized as a tissue transplant in all medical conditions where new tissue growth is desirable, and where it is desirable to minimize transplant tissue rejection.

The autologous or allogeneic ELA stem cells can be proliferated in an undifferentiated state through mitotic expansion in specific media, to obtain sufficient numbers of cells for use in the transplants by the methods described herein. See, Caplan and Haynesworth, U.S. Pat. Nos. 5,486,359; 5,197,985; and 5,226,914; and international patent application W092/22584. In another currently preferred aspect of the invention, ELA stem cells are prepared and used without further treatment or are purified, and do not need to be expanded in culture, i.e. in vitro, to obtain sufficient numbers of cells for use in the methods described herein. Thus, in a preferred embodiment, the human autologous ELA stem cells are obtained from the tissue or fluid of an individual donor as opposed to a pooled source of multiple donors. The subject human ELA stem cells are obtained from the tissue or fluid of a donor, exemplary tissues and fluids including blood, synovial fluid, and bone marrow. Thus, for example, donor human ELA stem cells are obtained from the patient per se or from another individual, respectively, and are introduced as a syngeneic autologous transplant or as an allogeneic transplant, respectively, into the recipient patient in need thereof, e.g. for skeletal repair. The donor and recipient are most likely to be allogeneic, and it is observed in examples herein that the transplant cell populations attenuate and seemingly eliminate a graft versus host response normally observed in allogeneic tissue transplants. Further, osteogenic properties of the ELA cell manifest in the microenvironment of the skeletal bones/vertebrae, and dermal, connective, sebaceous, vascular and neural tissues are repaired or regenerated in deep wounds, resulting e.g., from burns and trauma. In particular, the accelerated tissue healing properties of negative pressure treatment serve to induce robust tissue differentiation capabilities in ELA transplants, while maintaining an immune response attenuation with respect to the transplant but enhanced immune detection to pathogenic microorganisms and response to infection.

Similarly, ELA stem cells may be activated prior to transplant to induce and expedite their differentiation into neurons, fibroblasts, osteoblasts, chondrocytes, and various other types of tissues by a number of factors, including mechanical, cellular, and biochemical stimuli. Human ELA stem cells possess the potential to differentiate into cells such as adipocytes, osteoblasts and chondrocytes, which produce a wide variety of ELA tissue cells, as well as tendon, ligament and dermis, and this potential is retained after isolation and for several population expansions in culture. Thus, by being able to isolate, purify, greatly multiply, and then activate ELA stem cells to differentiate into the specific types of ELA cells desired, such as skeletal and connective tissues such as bone, cartilage, tendon, ligament, muscle, adipose and marrow stroma, see U.S. Pat. Application 61/247,236 a highly effective transplant exists for treating a wide variety of tissue transplant needs, including skeletal and other connective tissue disorders.

In an additional aspect, the present invention is directed to various methods of utilizing human autologous or allogeneic ELA stem or progenitor cells for therapeutic and/or diagnostic purposes. For example, autologous or allogeneic human ELA stem or progenitor cells find use in: (1) regenerating tissues which have been damaged through acute injury, abnormal genetic expression or acquired disease; (2) treating a host with damaged tissue by treatment of damaged tissue with autologous or allogeneic ELA stem cells combined with a biocompatible carrier suitable for delivering ELA stem cells to the damaged tissues site(s); (3) producing various tissues; (4) detecting and evaluating growth factors relevant to ELA stem cell self-regeneration and differentiation into committed lineages; (5) detecting and evaluating inhibitory factors which modulate ELA stem cell commitment and differentiation into specific ELA lineages; and (6) developing ELA cell lineages and assaying for factors associated with tissue development.

The dose of the autologous or allogeneic ELA stem cells varies within wide limits and will, of course be fitted to the individual requirements in each particular case. The number of ELA stem cells used as the transplant will depend on the size of the defect, the weight and condition of the recipient and other variables known to those of skill in the art. Generally, the ELA cells are used at lower concentrations for small defects, e.g., about 10,000-75,000 for cervical spine procedures, about 10K to 150K for lumbar spine procedures, about 30,000-1,000,000 or more for long bone procedures, about 10,000 to 500,000 for soft tissue remodeling, muscle and cardiac repair. Very generally, the ELA stem cells are use as a transplant at an application number from 1000 cells to about 5 million cells. However, in certain applications such as transplant of ELA stem cells transfected with a particular gene, the number of cells in the transplant may be substantially less, e.g., about one cell, 10 cells, 100 cells, 1000 cells or more. The ELA cells can be administered by any route that is suitable for the particular tissue or organ to be treated. The cells can be administered directly to the site of injury or site in need of repair or administered systemically, i.e., parenterally, by intravenous injection. In most cases, the autologous or allogeneic ELA stem cells are delivered to the site of desired treatment or therapy and can be targeted to a particular tissue or organ. The human ELA stem cells can be administered via a subcutaneous implantation of cells or by injection of stem cells, for example, into muscle cells, or by infusion, for cardiac repair.

The transplants include cells that are suspended in an appropriate diluent. Excipients for such solutions are biologically and physiologically compatible with the recipient, such as buffered saline solution. Other excipients may include water, isotonic common salt solutions, glycerin, DMSO or other cryoprotectants, alcohols, polyols, glycerine and vegetable oils. A currently preferred cellular transplant comprises about 10K to 200K cells in a buffered solution containing a cryoprotectant, which is provided frozen prior to transplant. The composition for administration is formulated, produced and stored according to standard methods complying with proper sterility and stability, and it is preferable to freeze the transplant on liquid nitrogen vapor or dry ice and thaw the transplant immediately prior to incorporation into the recipient.

In another aspect, the present invention relates to a method for repairing connective tissue damage. The method comprises the steps of applying an autologous or allogeneic ELA stem or progenitor cell-containing transplant to an area of tissue damage or an area requiring tissue growth, under conditions suitable for differentiating the cells into the type of connective tissue necessary for repair. In most transplant conditions, the particular tissue environment will be sufficient to cause differentiation of the ELA cells into their desired differentiated form.

In a further embodiment of this aspect, the present invention is directed to a method for enhancing the implantation of a prosthetic device into skeletal tissue. The method comprises the steps of adhering autologous or allogeneic ELA stem or progenitor cells onto the connective surface of a prosthetic device, and implanting the prosthetic device containing these ELA cells under conditions suitable for differentiating the cells into the type of skeletal or connective tissue needed for implantation.

The invention provides a transplant and a method for augmenting bone formation in an individual in need thereof. Thus, the methods of this aspect of the invention are applicable to “connective tissue defects” that include any damage or irregularity compared to normal connective tissue. Such damage or irregularity may occur due to trauma, disease, age, birth defect, or surgical intervention. More particularly, the invention provides a method for effecting the repair of segmental bone defects, nonunions, malunions or delayed unions. As used herein, “connective tissue defects” also refers to non-damaged areas in which bone formation is solely desired, for example, for cosmetic augmentation. The methods and materials disclosed herein are therefore suitable for use in orthopedic, dental, oral, maxillofacial, periodontal and other surgical procedures.

The present invention is also directed to transplants and methods of utilizing the autologous or allogeneic ELA progenitor or stem cells for correcting or modifying connective tissue disorders. Thus, in another aspect, the present invention is directed to various transplant devices and factors that have been developed in order to induce the autologous or allogeneic ELA stem or progenitor cells to differentiate into specific types of desired phenotypes, such as bone, fat or cartilage forming cells. For example, the inventors have found that various porous tri-calcium or hydroxyapatite ceramic devices can be utilized as vehicles or carriers for the autologous or allogeneic ELA stem cell transplants when implanted into skeletal defects thereby permitting and/or promoting the differentiation of the cells into skeletal tissue. See patent application 61/247,236.

Thus, one embodiment of the invention is directed to a transplant and a method for using a porous ceramic composition comprised of tri-calcium phosphate or hydroxyapatite or combinations of the two, as a vehicle or carrier for ELA stem or progenitor cells, which when implanted into skeletal defects, promotes the differentiation of the cells into skeletal tissue.

In another embodiment, the invention is directed to a transplant and a method for using absorbable gelatin, cellulose, and/or collagen-based matrix in combination with the autologous or allogeneic ELA stem cells. This transplant can be used in the form of a sponge, strip, powder, gel, web or other physical format.

In another embodiment, the invention is directed to a method for employing hyaluronic acid based transplants for the delivery of human ELA stem cells for repair of connective tissue or as a patch for the human ELA cells to allow for cartilage formation and repair.

Various alternative vehicles may be employed for delivery of human ELA transplants for repair of connective tissue. The compositions may be designed as a patch for the damaged tissue to provide bulk and scaffolding for new bone or cartilage formation. The various compositions, methods, and materials described herein can, in accordance with the present invention, be used to stimulate repair of fresh fractures, non-union fractures and to promote spinal fusion. See U.S. Pat. No. 5,197,985 for examples. Likewise, repair of cartilage and other musculoskeletal tissues can be accomplished. In the case of spinal fusion, such compositions, methods, and materials can be used posteriorly with or without instrumentation to promote mass fusion along the lamina and transverse processes and anteriorly, used to fill a fusion cage to promote interbody fusion. The methods of the present invention using autologous or allogeneic ELA transplants can be used to treat total joint replacement and osteoporosis.

In accordance with another aspect of the invention, the ELA transplants can be used to produce marrow stroma. The marrow stroma provides the scaffolding as well as soluble factors which direct and support blood cell synthesis, i.e., hematopoiesis. Accordingly, this aspect of the invention is directed to a method to improve the process of blood cell and marrow tissue regeneration in patients where the marrow is depleted or destroyed, such as, for example, during intensive radiation and chemotherapy treatment, by employing hematopoietic progenitor cells derived from, for example, bone marrow or peripheral blood.

Accordingly, in one embodiment, the present invention provides transplants and methods of making and using autologous or allogeneic ELA transplants to enhance engraftment of hematopoietic stem or progenitor cells. Thus, one embodiment of the present invention provides a method for enhancing the regeneration of marrow tissue by using autologous or allogeneic ELA stem cells. The method for enhancing hematopoietic stem or progenitor cell engraftment comprises administering to an individual in need thereof, (i) autologous or allogeneic ELA stem cells and (ii) hematopoietic stem or progenitor cells, wherein said ELA stem cells are administered in an amount effective to promote engraftment of such hematopoietic stem or progenitor cells in the individual. More particularly, one embodiment of the invention is directed to a method for using ELA stem cells which, when administered systemically, will migrate, or home, to the marrow cavity and differentiate into marrow stroma, thereby regenerating the marrow stroma. The autologous or allogeneic ELA stem cells can be administered systemically, e.g., intravenously, into various delivery sites or directly into the bone.

A further consideration in this aspect is directed to the timing of injection of the autologous or allogeneic ELA transplants into the patient relative to the administration of hematopoietic stem or progenitor cells. In one embodiment, the ELA transplants are injected simultaneously with the hematopoietic stem or progenitor cells. In another embodiment, the ELA stem cells are administered before or after the administration of the hematopoietic stem or progenitor cells. The hematopoietic stem cells may be autologous or may be matched to the autologous or allogeneic cells.

The present invention is useful to enhance the effectiveness of bone marrow transplantation as a treatment for cancer or nuclear radiation poisoning. The treatment of cancer by x-irradiation or alkylating therapy destroys the bone marrow microenvironment as well as the hematopoietic stem cells. The current treatment is to transplant the patient after marrow ablation with bone marrow which has been previously harvested and cryopreserved. However, because the bone marrow microenvironment is destroyed, bone marrow engraftment is delayed until the stromal environment is restored. As a result, an aspect of the present invention is directed to the advantages of transplanting non-expanded or culture-expanded autologous or allogeneic ELA stem cells to accelerate the process of stromal reconstitution and regeneration of marrow tissue.

Modes of administration of the ELA transplant include but are not limited to systemic intravenous injection or injection directly to the intended site of activity. The preparation can be administered by any convenient route, for example by infusion or bolus injection and can be administered together with other biologically active agents.

In this aspect of the invention, the ELA transplant can be administered alone, however in an alternative embodiment, the ELA stem cells are utilized in the form of pharmaceutically accepted transplants. Such transplants comprise a therapeutically effective amount of the autologous or allogeneic ELA stem cells, and a pharmaceutically acceptable carrier or excipient. Such a carrier includes but is not limited to saline, buffered saline, dextrose, water, and combinations thereof. The formulation should suit the mode of administration. In this embodiment, the ELA transplant is formulated in accordance with routine procedures as a pharmaceutical composition adapted for intravenous administration to human beings. Typically, compositions for intravenous administration are solutions in sterile isotonic aqueous buffer. Where necessary, the composition may also include a local anesthetic to ameliorate any pain at the site of the injection. Generally, the ingredients are supplied either separately or mixed together in unit dosage form, for example, as a cryopreserved concentrate in a hermetically sealed container such as an ampoule indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The transplants and methods of the invention can be altered, particularly by (1) increasing or decreasing the time interval between implanting the ELA transplant and implanting the tissue; (2) increasing or decreasing the amount of ELA stem cells injected; (3) varying the number of ELA stem cell injections; or (4) varying the method of delivery of ELA transplant.

The ELA transplant is used in an amount effective to promote engraftment of for example hematopoietic or osteoprogenitor cells in the recipient. In general, such amount is at least ten thousand ELA stem cells and most generally need not be more than five million ELA stem cells/kg. Preferably, it is at least about fifty thousand ELA stem cells and usually need not be more than about two million ELA stem cells. The autologous or allogeneic ELA transplant may be administered concurrently with other transplant cells such as transfused blood.

The invention also provides a pharmaceutically packaged therapeutic transplant kit comprising one or more containers filled with a preparation of ELA stem cells for transplant. Associated with such container(s) can be a notice in the form prescribed by a governmental agency regulating the manufacture, use or sale of pharmaceuticals or biological products, which notice reflects approval by the agency of manufacture, use or sale for human administration, as well as instructions for using the transplant cells. A preferred embodiment is a cryopreserved preparation of the ELA stem cell transplant. The transplant is stored frozen under liquid nitrogen vapor, and shipped in a cryocontainer or on dry ice, to a surgeon for use in a surgical procedure. The packaging of the transplant provides for a sterile field in accordance with surgical techniques.

The present invention is particularly advantageous in that ELA stem cell transplant may be used for a variety of treatments wherein the source of the ELA stem cells is other than the recipient and without requiring that such source be matched to the recipient. Moreover, such allogeneic ELA transplants may be used without requiring chronic administration of immunosuppressants, as the ELA cells downregulate the cell mediated and humoral self-immune responses and transplanted tissues do not exhibit typical graft versus host responses commonly seen with allografts. More advantageously, the immune cells in an ELA transplant survey the transplant site and provide an enhanced immune response against pathogenic microorganisms and greater infection prevention. Contrast this with typical allografts that require immunodepleting drugs, leaving a patient vulnerable to infection.

In a still further aspect, the present invention also is directed to the application of autologous or allogeneic human ELA transplant having genetically engineered cells that carry within them genes of interest particularly for the expression of physiologically or pharmacologically active proteins or for use in gene therapy. In accordance with this aspect of the present invention, autologous or allogeneic human ELA transplants can be used as host cells for the expression of exogenous gene products. These culture-expanded cells home to the marrow and enhance hematopoietic recovery in a marrow transplant setting. Furthermore, these cells can be manipulated for cellular therapy, e.g. expanded, purified, selected and maintained for clinical use while still maintaining their precursor phenotype. Part of this manipulation is the characterization of such cells and their cryopreservation for future use. It is contemplated that the transformed autologous or allogeneic stem cell transplants and the expression products of the incorporated genetic material can be used alone or in combination with other cells and/or compositions.

The technology used to introduce foreign genes into progenitor cell cultures has been described, e.g. see U.S. Pat. No. 5,591,625, and provides transduced stem cells wherein all progeny of the cells carry the new genetic material. Cell delivery of the transformed cells can be effected through various modes including infusion and direct injection into periosteal, bone marrow, muscle and subcutaneous sites.

By virtue of this aspect of the present invention, genes can be introduced into autologous or allogeneic ELA transplants which are then administered to the patient where gene expression will effect its therapeutic benefit Examples of such applications include genes that have a central role in ELA cell maintenance, tissue development, remodeling, repair and in vivo production of extracellular gene products.

In addition to the correction of genetic disorders, this aspect of the present invention can introduce, in a targeted manner, additional copies of essential genes to allow augmented expression of certain gene products. These genes can be, for example, hormones, matrix proteins, cell membrane proteins cytokines, adhesion molecules, detoxification enzymes and “rebuilding” proteins important in tissue repair. Normal ELA transplants are accordingly used to treat abnormal ELA stem cells.

An additional application is the use of introduced genes to alter the phenotype of the autologous or allogeneic ELA stem cells and their differentiated progeny for specific therapeutic applications. This includes intracellular gene products, signal transduction molecules, cell surface proteins, extracellular gene expression products and hormone receptors. Disease states and procedures for which such treatments have application include genetic disorders of the musculoskeletal system, diseases of bone and cartilage, the bone marrow, inflammatory conditions, muscle degenerative diseases, malignancies and autologous or allogeneic bone or bone marrow transplantation.

In one embodiment, the human ELA transplant preferably includes ELA stem cells that have been transformed with at least one DNA sequence capable of expressing those translation products capable of packaging a viral sequence so as to be gene therapy producer cells. In a preferred embodiment of this aspect, the human ELA transplants have been transformed with a DNA sequence comprising a retroviral 5′ LTR and, under the transcriptional control thereof, at least one of a retroviral gag, pol or env gene. In another aspect, the ELA transplant contains cells transformed with a DNA sequence comprising a retroviral packaging signal sequence and incorporated genetic material to be expressed under the control of a promoter therefore so as to be incompetent retroviruses. Also contemplated is a transplant having transfected ELA stem cells to initiate, modulate or augment hematopoiesis.

Virtually all genetic lesions can be treated, for example using autologous ELA cells or tissue can be treated or “corrected” by technology involving incorporation of genetic material. A key component is the ability to deliver these gene-carrying stem cells to the proper tissue under the conditions that the stem cells will expand and repopulate the tissue space. Patient preparation for introduction of autologous or allogeneic ELA stem cells includes, but is not limited to, (a) marrow ablation by chemotherapy and/or irradiation in conjunction with marrow transplantation, and (b) direct tissue infiltration of “transduced” cells without preparation, particularly where the transduced cells might have a survival advantage, an advantage during differentiation or an advantage in function (such as might be the case when correcting a muscle disorder such as muscular dystrophy with the dystrophin or similar gene). An additional application is in the tagging of ELA stem cells prepared for use in vivo alone or as applied to any indwelling device, such as, for example, an orthopedic device in which it is of interest to “mark” the ELA stem cells and observe their survival, maintenance and differentiation and their association with the device over time.

The advantages provided by an autologous or an allogeneic human ELA transplant transfected with exogenous genetic material encoding a protein to be expressed include the ability to utilize human ELA stem cells obtained from the same individual i.e., autologous to the individual or a variety of sources other than the individual into which they will be administered, i.e., allogeneic to the individual; the ability to deliver these gene-carrying ELA stem cells to the proper tissue in a patient without inducing an adverse immune response, thus minimizing the need for immunosuppressive therapy prior to administration of the cells; the ability to culturally expand human ELA stem cells for infusion where they will localize to other tissue spaces; the ability to culturally expand and cryopreserve human ELA stem cells which can be used as hosts for stable, heritable gene transfer; the ability to recover genetically altered cells after installation in vivo; the ability to match a genetic therapy to a wide variety of disorders, pinpointing the genetic alteration to the target tissue; and the ability of newly introduced genes within human ELA stem cells and their progeny to be expressed in a less restrictive fashion than other cells, thereby expanding the potential application in treating medical disease.

The structure and life cycle of retroviruses makes them ideally suited to be gene-transfer vehicles. Generally regarding retroviral mediated gene transfer, see McLachlin et al., Progress in Nucleic Acid Research and Molecular Biology, 38:91-135 (1990). Transformation of stem cells using retroviruses has been described in U.S. Pat. No. 5,591,625.

It is also possible to use vehicles other than retroviruses to genetically engineer or modify the autologous or allogeneic transplant. Genetic information of interest can be introduced by means of any virus which can express the new genetic material in such cells, for example, SV40, herpes virus, adenovirus and human papillomavirus. Many methods can be used for introducing cloned eukaryotic DNAs into cultured mammalian cells, which include transfection mediated by either calcium phosphate or DEAE-dextran, protoplast fusion and electroporation. The genetic material to be transferred to transplant may be in the form of viral nucleic acids, bacterial plasmids or episomes.

The present invention makes it possible to genetically engineer autologous or allogeneic ELA transplants in such a manner that they produce polypeptides, hormones and proteins not normally produced in human stem cells in biologically significant amounts or produced in small amounts but in situations in which overproduction would lead to a therapeutic benefit. While human ELA stem cells are a preferred embodiment, xenogeneic ELA cells are also within the scope herein, for example, porcine, bovine, and equine ELA cells. The transplant proteins are secreted into the bloodstream or other areas of the body, such as the central nervous system. The proteins formed in this way can serve as a continuous drug delivery systems to replace present regimens, which require periodic administration (by ingestion, injection, depot infusion etc.) of the needed substance. This invention has applicability in providing hormones, enzymes and drugs to humans and to high value animals, in need of such substances. It is particularly valuable in providing such substances, such as hormones (e.g., parathyroid hormone, insulin), which are needed in sustained doses for extended periods of time. For example, it can be used to provide continuous delivery of insulin, and, as a result, there would be no need for daily injections of insulin. Genetically engineered ELA transplants can also be used for the production of clotting factors such as Factor VIII, or for continuous delivery of dystrophin to muscle cells for muscular dystrophy.

Incorporation of genetic material of interest into autologous or allogeneic ELA transplants is particularly valuable in the treatment of inherited and acquired disease. In the case of inherited diseases, this approach is used to provide genetically modified ELA transplants which can be used as a metabolic sink. That is, such ELA transplants would serve to degrade a potentially toxic substance. For example, this could be used in treating disorders of amino acid catabolism including the hyperphenylalaninemias, due to a defect in phenylalanine hydroxylase; the homocysteinemias, due to a defect in cystathionine beta-synthase. Other disorders that could be treated in this way include disorders of amino acid metabolism, such as cystinosis; disorders of membrane transport, such as histidinurea or familial hypocholesterolemia; and disorders of nucleic acid metabolism, such as hereditary orotic aciduria.

Autologous or allogeneic or xenogeneic ELA transplants of the present invention can also be used in the treatment of genetic diseases in which a product (e.g., an enzyme or hormone) normally produced by the body is not produced or is made in insufficient quantities. Here, ELA stem cells transduced with a gene encoding the missing or inadequately produced substance can be used to produce it in sufficient quantities. This can be used in producing alpha-1 antitrypsin. It can also be used in the production of Factor XIII and Factor IX and thus would be useful in treating hemophilia.

There are many acquired diseases for which treatment can be provided through the use of engineered autologous or allogeneic or xenogeneic ELA transplants (e.g., human ELA stem cells transduced with genetic material of interest). For example, such cells can be used in treating anemia, which is commonly present in chronic disease and often associated with chronic renal failure (e.g., in hemodialysis patients). In this case a transplant having for example human ELA stem cells having incorporated in them a gene encoding erythropoietin would correct the anemia by stimulating the bone marrow to increase erythropoiesis (i.e. production of red blood cells). Other encoded cytokines can be G-CSF or GM-CSF, for example.

The autologous or allogeneic ELA transplants of the present invention can also be used to administer a low dose of tissue plasminogen activator (TPA) as an activator to prevent the formation of thrombin. For example, human ELA stem cells having incorporated genetic material which encodes TPA would be placed into a transplant for an individual in whom thrombus prevention is desired. This would be useful, for example, as a prophylactic against common disorders, such as coronary artery disease, cerebrovascular disease, peripheral vascular occlusive disease, vein (e.g., superficial) thrombosis, such as seen in pulmonary emboli, or deep vein thrombosis. An ELA transplant which contain DNA encoding calcitonin can be used in the treatment of Paget's Disease, a progressive, chronic disorder of bone metabolism, in which calcitonin is presently administered subcutaneously.

Another application is a subcutaneous implantation of an autologous or allogeneic or xenogeneic ELA transplant with cells adhered to a porous ceramic cube device which will house the ELA stem cells and allow them to differentiate in vivo. Another example would be injection of an autologous or allogeneic or xenogeneic ELA transplant into muscle to differentiate into muscle cells. Another example might be a graft having genetically engineered ELA stem cells which continuously secrete a polypeptide hormone, e.g. luteinizing hormone-releasing hormone (LHRH) for use in birth control.

ELA transplants engineered to produce and secrete interleukins (e.g., IL-1, IL-2, IL-3 or IL-4 through IL-11) can be used in several contexts. For example, administration of IL-3 through an ELA transplant which contains genetic material encoding IL-3 can be used to increase neutrophil count to treat neutropenia. Autologous or allogeneic or xenogeneic ELA transplants can also be transduced with the gene for thrombopoietin and when administered to an individual having a condition marked by a low platelet count, production and secretion of the encoded product will result in stimulation of platelet production.

Another use of the present invention is in the treatment of enzyme defect diseases. In this case the product (polypeptide) encoded by the gene introduced into human ELA stem cells is not secreted (as are hormones); rather, it is an enzyme that remains inside the cell. There are numerous cases of genetic diseases in which an individual lacks a particular enzyme and is not able to metabolize various amino acids or other metabolites. The correct genes for these enzymes could be introduced into the autologous or allogeneic ELA stem cells and transplanted into the individual; the transplant would then carry out that metabolic function. For example, there is a genetic disease in which those affected lack the enzyme adenosine deaminase. This enzyme is involved in the degradation of purines to uric acid. It is believed possible, using the present invention, to produce a subcutaneous graft as described above capable of producing the missing enzyme at sufficiently high levels to detoxify the blood as it passes through the area to which the graft is applied.

Additional uses include but are not limited to cytokine genes to enhance hematopoietic reconstitution following marrow transplantation for bone marrow failure for congenital disorders of the marrow; bone cytokines to improve repair and healing of injured bone; bone matrix problems to improve repair and healing of injured or degenerative bone; correction of ELA genetic disorders such as osteogenic imperfecta and muscular dystrophy; localized production of proteins, hormones etc. providing cellular therapeutics for many different compounds; and cytotoxic genes such as thymidine kinase which sensitizes cells to gangiclovir. Gap junction adhesion to tumor cells could allow ELA stem cells to serve for cancer therapy.

Bone grafting procedures are widely used to treat acute fractures, fracture non-unions, bone defects, and to achieve therapeutic arthrodesis. Autogenous cancellous bone is the current “gold standard” for clinical bone grafting. Contemporary dogma attributes this effectiveness to three primary intrinsic properties: osteoconduction, osteogenic cells, and osteoinduction.

Transplants that include ELA stem cells can be autologous, allogeneic or from xenogeneic sources. ELA stem cells may be obtained from synovial fluid, blood, bone marrow, tissue and other fluids in the body. ELA transplants are obtained by providing a bodily fluid from a subject (e.g., a mammal such as a human; enriching for a population of ELA stem cells; and may optionally include depleting cells from the population expressing stem cells surface markers, thereby isolating a population of ELA stem cells e.g., as described in U.S. provisional patent application Ser. No. 60/927,596 hereby incorporated herein by reference in its entirety.

Transplants are provided for the repair of bone defects by the rapid regeneration of healthy bone. The transplant may include an absorbable gelatin, cellulose and/or collagen-based matrix or a resorbable biopolymer or matrix selected from the group consisting of a natural or synthetic matrix, e.g. demineralized bone, allograft, autograft, oxygen carrying hydrogel, gelatin, collagen, cellulose, or bone graft synthetic substitute, e.g. beta-tricalcium phosphate scaffold or other commercially or non-commercially available biopolymer or matrix in combination with stem cells. The transplant can be manufactured in the form of a sponge, strip, putty, powder, gel, web, liquid or other physical format. The transplant is, for example, inserted in the defect and results in osteogenic healing of the defect.

The transplant can also contain additional components, such as osteoinductive factors. Such osteoinductive factors include, for example, dexamethasone, ascorbic acid-2-phosphate, beta-glycerophosphate and TGF-β, super-family proteins, such as the bone morphogenic proteins (BMPs). The transplant can also contain antibiotic, antimycotic, antiinflammatory, immunosuppressive and other types of therapeutic, preservative and excipient agents.

The invention also provides a method for treating a bone defect in an animal, particularly a mammal and even more particularly a human, in need thereof, which comprises administering to the bone defect of said animal a bone defect-regenerative amount of the transplant of the invention.

The invention also provides for the in vivo healing potential of a transplant that contains freshly prepared or cryopreserved, unexpanded, culture expanded or master cell bank generated ELA stem cells inserted in the defect area alone.

The invention also provides for the in vivo healing potential of a transplant that contains freshly prepared or culture expanded ELA stem cells delivered in the matrix alone.

The invention also contemplates the use of other extracellular matrix components, along with the cells, so as to achieve osteoconductive or osteoinductive properties. In addition, by varying the ratios of the components in said biodegradable matrices, surgical handling properties of the cell-biomatrix implants can be adjusted in a range from a dimensionally stable matrix, such as a sponge or film, to a moldable, putty-like consistency to a pliable gel or slurry to a powder.

In an embodiment, the transplant of the invention comprises an absorbable support, containing ELA stem cells for repair of segmental defects, spinal fusions or non-unions and other bone defects. Custom cell-matrix implants containing autologous, allogeneic or xenogeneic ELA stem cells can be administered using open surgical techniques, arthroscopic techniques or percutaneous injection.

Human ELA stem cells can be provided for the transplant as freshly prepared or cryopreserved, non-expanded, culture-expanded or master cell bank generated preparations derived from human sources of autologous or allogeneic or xenogeneic ELA stem cells or from ELA stem cell-enriched or heterogenous cultures containing an effective dose of at least about 10² and preferably about 10⁵, preferably about 10⁴ or up to about 10⁶, ELA stem cells per milliliter of the composition. For effective clinical outcomes, in this embodiment using an ELA stem cell transplant that number of freshly prepared or cryopreserved, unexpanded, expanded, or master cell bank generated ELA stem cells is provided to the patient, or about the same number in an optimized medium, which repairs the bone or other tissue defect. This is referred to as the “Regenerative ELA Stem Cell Threshold”, or that concentration of ELA stem cells necessary to achieve direct repair of the tissue defect. The Regenerative ELA Stem Cell Threshold will vary by: type of tissue (i.e., bone, cartilage, ligament, tendon, muscle, marrow stroma, dermis and other connective tissue); source of tissue (i.e., syngeneic, allogeneic, xenogeneic); degree of desired immune attenuation and/or immune surveillance; size or extent of tissue defect; formulation with pharmaceutical carrier; age of the patient; type of matrix and bioactive factors.

In an embodiment, the method further comprises administering at least one bioactive factor, which further induces or accelerates the differentiation of the ELA transplant into the osteogenic lineage. Preferably, the cells are contacted with the bioactive factor ex vivo, while in the matrix, or injected into the defect site at or following the implantation of the composition of the invention. It is particularly preferred that the bioactive factor is a member of the TGF-β superfamily comprising various tissue growth factors, bioactive glass, particularly bone morphogenic proteins, such as at least one selected from the group consisting of BMP-2, BMP-3, BMP-4, BMP-6 and BMP-7.

In the embodiment which uses a gelatin-based matrix, an appropriate absorbable gelatin sponge, powder or film is cross-linked gelatin, for example, Gelfoam. (Upjohn, Inc., Kalamazoo, Mich.) which is formed from denatured collagen. The absorbable gelatin-based matrix can be combined with the bone reparative cells and, optionally, other active ingredients by soaking the absorbable gelatin sponge in a cell suspension of the ELA stem cells, where the suspension liquid can have other active ingredients dissolved therein. Alternately, a predetermined amount of a cell suspension can be transferred on top of the gelatin sponge, and the cell suspension can be absorbed.

In the embodiment that uses a cellulose-based matrix, an appropriate absorbable cellulose is regenerated oxidized cellulose sheet material, for example, Surgicel (Johnson & Johnson, New Brunswick, N.J.) which is available in the form of various sized strips or OXYCEL® (Becton Dickinson, Franklin Lakes, N.J.) which is available in the form of various sized pads, pledgets and strips. The absorbable cellulose-based matrix can be combined with the bone reparative cells and, optionally, other active ingredients by soaking the absorbable cellulose-based matrix in transplant made with a cell suspension of the ELA stem cells, and the suspension liquid has other active ingredients dissolved therein. Alternately, a predetermined amount of a cell suspension is transferred to the top of the cellulose-based matrix, and the cell suspension is absorbed to form the transplant.

In the embodiment which uses a collagen-based matrix, an appropriate resorbable collagen is purified bovine corium collagen, for example, Avitene (MedChem, Woburn, Mass. which is available in various sizes of nonwoven web and fibrous foam, HELISTAT® (Marion Merrell Dow, Kansas City, Mo.) which is available in various size sponges or HEMOTENE®. (Astra, Westborough, Mass.) which is available in powder form. The resorbable collagen-based matrix is combined with the bone reparative cells and, optionally, other active ingredients by soaking the resorbable collagen-based matrix in a cell suspension of the ELA stem cells to form the transplant, and the suspension liquid can have other active ingredients dissolved therein. Alternately, a predetermined amount of a cell suspension is transferred on top of the collagen-based matrix, and the cell suspension can be absorbed.

The above gelatin-based, cellulose-based and collagen-based matrices may, optionally, possess hemostatic properties.

Preferred active ingredients are those biological agents, which enhance wound healing or regeneration of bone, particularly recombinant proteins. Such active ingredients are present in an amount sufficient to enhance healing of a wound, i.e., a wound healing-effective amount. The actual amount of the active ingredient will be determined by the attending clinician and will depend on various factors such as the severity of the wound, the condition of the patient, the age of the patient and any collateral injuries or medical ailments possessed by the patient. Generally, the amount of active ingredient will be in the range of about 1 μg/cm³ to 5 mg/cm³.

Implantation of a transplant containing unexpanded or culture-expanded autologous or allogeneic or xenogeneic ELA stem cell populations offers the advantage of directly delivering the cellular machinery responsible for synthesizing new bone, and circumventing the otherwise slow steps leading to bone repair. Even in patients with a reduced ability to regenerate connective tissue, presumably due to a low titer of endogenous mesenchymal stem cells (Kahn et al. 1995 Clin. Orthop. Rel. Res. 313:69-75, Tabuchi et al. 1986 J. Clin. Invest. 78:637-642, Werntz et al. 1996 J. Orthop. Res. 14:85-93, Bruder et al. 1994 J. Cell. Biochem. 56:283-294), the ELA stem cells may be obtained and implanted without culture expansion and restore or enhance the patient's ability to heal tissue defects. In an alternative embodiment, the ELA stem cells may be prepared and culture-expanded over one billion-fold without a loss in their osteogenic potential, thus restoring or enhancing a patient's ability to heal tissue defects.

Synovial fluid includes various types of mononuclear cells and the ELA cell. In general, the non ELA cells are immune cells which include T cells, B cells, NK cells, monocytes, and dendritic cells. The monocytes, T, B, and NK cells make up less than 5% respectively, while the dendritic cells make up in most cases, greater than 50% of the immune cells population.

Dendritic cells (DC) include myeloid (mDC) and plasmacytoid (pDC) dendritic cells. The myeloid dendritic cell is instrumental in the induction of T cells activation and the plasmocytoid DC suppresses T cell activation. The plasmacytoid dendritic cell is a rare population of immune cells generally found in peripheral blood and in tissues. Surprisingly, the cells are found at extremely large numbers in synovial fluid, and in even greater amounts in synovial fluid from osteoarthritic donors. These cells express the surface marker CD123, CD14, and CD2. Upon stimulation and subsequent activation, these cells produce large amounts of type I interferon (mainly IFN-α and IFN-β), which are important pleiotropic anti-viral compounds mediating a wide range of effects of which one is suppression T cell activation.

Without being limited by any particular theory or mechanism of action, it is here envisioned that the pDC is responsible for orchestrating a regenerative immune response and suppressing the defensive immune response. Both mDCs and pDCs express the delta ligand, which binds the notch receptor. The interaction of these molecules augments mitogenic activity. It is likely that delta ligand located on the surface of mDC are involved in efficient activation of T cells and that the delta ligand on pDC is responsible for efficient activation of the ELA cell.

In the aggregate synovial fluid mononuclear cells population, the major cell component are ELA cells and the next most plentiful cell is the pDC. In addition to the previous noted cells, the transplants provided herein contain microparticles and platelets, which may play a role in tissue regeneration. Microparticles are small vesicles of heterogeneous density and size released by circulating blood cells and endothelial cells. Among other things, such microparticles have been described for a variety of cell types such as platelets (PMP, platelet microparticles) (Horstman and Ahn, 1999; Nomura, 2001), endothelial cells (EMP, endothelial cell microparticles) (Brogan and Dillon, 2004; Horstman et al, 2004), granulocytes (GMP, granulocyte microparticles), erythrocytes. These particles circulate in the blood and may affect various biological functions of target cells by direct interaction. Cellular microparticles bear at least some antigenic markers derived from their cells of origin and also contain cytoplasmic components of the original cell (Freyssinet, 2003; Horstman et al 2004). Their release is triggered by a variety of conditions, including cell activation, cell death by apoptosis, partial or complete complement lysis, oxidative stress, physical stress such as shear stress. Microparticles are generally considered to be a sign of cellular dysfunction and serve as general indicators of cell injury, stress, thrombosis, and inflammation. A variety of mediators of inflammation, coagulation and clotting factors, angiogenic factors, growth factors, cell surface antigens are bound to microparticles and microparticles thus may perform yet unappreciated signaling functions under normal and pathophysiological conditions (Morel et al, 2004; Horstman et al, 2004; Hugel et al, 2005; Martinez et al, 2005; Eilertsen and Osterud, 2005; Brogan and Dillon, 2004; Freyssinet et al, 2003).

Exosomes are derived from intracellular multivesicular bodies through fusion of multivesicular late endosomes/lysosomes with the plasma membrane and are also released by platelets, leukocytes, and other cell types. They can be considered a distinct species of microparticles. They can present antigens and can perform and other functions (Horstman et al, 2004; Denzer et al, 2000). O′Neill and Quah (2008) have reported that exosomes derived from bacterially infected macrophages carry bacterial coat components and use these to stimulate bystander macrophages and neutrophils to secrete pro-inflammatory mediators, including TNF-alpha, the chemokine CCLS, and inducible nitric oxide synthase.

It is here envisioned that a unique group of microparticles in the synovial fluid accompany the ELA cells and pDC in the transplants provided herein. The presence of these particles further distinguish the population of cells including ELA cells from previously described adult early lineage cells for the purposes of improved transplants and methods of transplantation.

### USES AND METHODS

A population of ELA stem cells or an ELA stem cell transplant can be used as freshly prepared, or can be proliferated and expanded by culture. For example, the population of ELA stem cells can be cultured in tissue culture containers, e.g., dishes, flasks, multiwell plates, or the like, for a sufficient time for the stem cells to proliferate to 70-90% confluence, that is, until the stem cells and their progeny occupy 70-90% of the culturing surface area of the tissue culture container.

ELA stem cell populations can be seeded in culture vessels at a density that allows cell growth. For example, the cells may be seeded at low density (e.g., about 1,000 to about 5,000 cells/cm²) to high density (e.g., about 50,000 or more cells/cm²). In a preferred embodiment, the cells are cultured at about 0 to about 5 percent by volume CO₂ in air. In some preferred embodiments, the cells are cultured at about 2 to about 25 percent 0₂ in air, preferably about 5 to about 20 percent 0₂ in air. The cells preferably are cultured at about 25 C to about 40 C, preferably 37 C. The cells are preferably cultured in an incubator. The culture medium can be static or agitated, for example, using a bioreactor. ELA stem cells preferably are grown under low oxidative stress (e.g., with addition of glutathione, ascorbic acid, catalase, tocopherol, N-acetylcysteine, or the like).

Once 70%-90% confluence is obtained, the cells may be passaged. For example, the cells can be enzymatically treated, e.g., trypsinized, using techniques well-known in the art, to separate them from the tissue culture surface. After removing the cells by pipetting and counting the cells, about 20,000-100,000 stem cells, preferably about 50,000 stem cells, are passaged to a new culture container containing fresh culture medium. Typically, the new medium is the same type of medium from which the stem cells were removed. The invention encompasses populations of ELA stem cells that have been passaged at least about 1, 2, 3, 4, 5, 6, 7, 8, 9, 10, 12, 14, 16, 18, or 20 times, or more.

The growth of the stem cells for the transplants of the invention described herein, as for any mammalian cell, depends in part upon the particular medium selected for growth. Under optimum conditions the stem cells of the invention typically double in number in about 24 hours to about 4 days. Populations of the stem cells of the invention when cultured under appropriate conditions form embryoid-like bodies or colony forming units, that is, three-dimensional clusters of cells that express the embryonic form of Oct4 protein.

The stem cells can be seeded in culture vessels at a density that allows cell growth. For example, the cells may be seeded at low density (e.g., about 1,000 to about 5,000 cells/cm²) to a high density (e.g., about 50,000 or more cells/cm²).

ELA stem cell populations can be used to initiate, or seed, cell cultures. Cells are generally transferred to sterile tissue culture vessels either uncoated or coated with extracellular matrix or ligands such as laminin, collagen (e.g., native or denatured), gelatin, fibronectin, ornithine, vitronectin, and extracellular membrane protein (e.g., MATRIGEL® (BD Discovery Labware, Bedford, Mass.)). Preferably, proliferative stem cells of the invention are plated in fibronectin-coated wells of 96 well plates in defined medium consisting of 1% PHS, 10 ng/ml IGF, 10 ng/ml EGF and 10 ng/ml PDGF-BB as well as transferrin, selenium, dexamethasone, linoleic acid, insulin, and ascorbic acid.

ELA stem cells can be cultured in any medium, and under any conditions, recognized in the art as acceptable for the culture of stem cells. Preferably, the culture medium comprises serum. ELA stem cells can be cultured in, for example, DMEM-LG (Dulbecco's Modified Essential Medium, low glucose)/MCDB 201 (chick fibroblast basal medium) containing ITS (insulin-transferrin-selenium), LA+B SA (linoleic acid-bovine serum albumin), dextrose, L-ascorbic acid, PDGF, EGF, IGF-1, and penicillin/streptomycin; DMEM-HG (high glucose) comprising 10% fetal bovine serum (FBS); DMEM-HG comprising 15% FBS; IMDM (Iscove's modified Dulbecco's medium) comprising 10% FBS, 10% horse serum, and hydrocortisone; M199 comprising 10% FBS, EGF, and heparin; MEM (minimal essential medium) comprising 10% FBS, GLUTAMAX and gentamicin; DMEM comprising 10% FBS, GLUTAMAX and gentamicin, etc. A preferred medium is DMEM-LG/MCDB-201 comprising 2% FBS, ITS, LA+BSA, dextrose, L-ascorbic acid, PDGF, EGF, and penicillin/streptomycin. For example, the cells can be maintained in Dulbecco Minimal Essential Medium (DMEM) or any other appropriate cell culture medium, supplemented with 1-50 ng/ml (e.g., about 5-15 ng/ml) platelet-derived growth factor-BB (PDGF-BB), 1-50 ng/ml (e.g., about 5-15 ng/ml) epidermal growth factor (EGF), 1-50 ng/ml (e.g., about 5-15 ng/ml) insulin-like growth factor (IGF), or 100-10,000 IU (e.g., about 1,060) LIF, with 10⁻¹⁰ to 10⁻⁸M dexamethasone or other appropriate steroid, 2-10 μg/ml linoleic acid, and 0.05-0.15 μm ascorbic acid. Additional culture conditions can be identified by one of skill in the art.

In one example, about 50,000 cells are grown under suitable conditions. The cells can be plated in fibronectin-coated wells of 96 well plates in defined medium consisting of 1% PHS, 10 ng/ml IGF, 10 ng/ml EGF and 10 ng/ml PDGF-BB as well as transferrin, selenium, dexamethasone, linoleic acid, insulin, and ascorbic acid. The negatively-selected samples, which can optimally comprise a population of cells that is greater than 98% class I and glycophorin negative, can then be assessed for expression of adult and embryonic stem cell markers, as well as the lack of expression of MHC class I, MHC class II, CD44, CD45, CD13, CD34, CD49c, CD73, CD105, and CD90 cell surface markers, according to methods known in the art to confirm the identity of the purified population.

In specific embodiments, pooled human serum (1-2%) and human growth factors are used to supplement growth and proliferation. Preferably, stem cells of the invention are grown in the presence of 1-2% pooled human serum, epidermal growth factor, and platelet-derived growth factor-BB.

Other media in that can be used to culture ELA stem cells include DMEM (high or low glucose), Eagle's basal medium, Ham's F10 medium (F10), Ham's F-12 medium (F12), Iscove's modified Dulbecco's medium, Mesenchymal Stem Cell Growth Medium (MSCGM), Liebovitz's L-15 medium, MCDB, DMEM/F12, RPMI 1640, advanced DMEM (Gibco), DMEM/MCDB201 (Sigma), and CELL-GRO FREE.

Other appropriate media include, for example, Minimal Essential Medium (MEM), IMDM, and RPMI. Minimum Essential Medium (MEM) is one of the most widely used of all synthetic cell culture media. Early attempts to cultivate normal mammalian fibroblasts and certain subtypes of HeLa cells revealed that they had specific nutritional requirements that could not be met by Eagle's Basal Medium (BME). Subsequent studies using these and other cells in culture indicated that additions to BME could be made to aid growth of a wider variety of fastidious cells. MEM, which incorporates these modifications, includes higher concentrations of amino acids so that the medium more closely approximates the protein composition of mammalian cells. MEM has been used for cultivation of a wide variety of cells grown in monolayers. Optional supplementation of non-essential amino acids to the formulations that incorporate either Hanks' or Eagles' salts has broadened the usefulness of this medium. The formulation has been further modified by optional elimination of calcium to permit the growth of cells in suspension.

Iscove's Modified Dulbecco's Media (IMDM) is a highly enriched synthetic media. IMDM is well suited for rapidly proliferating, high-density cell cultures.

MCDB media were developed for the low-protein and serum free growth of specific cell types using hormones, growth factors, trace elements and/or low levels of dialyzed fetal bovine serum protein (FBSP). Each MCDB medium was formulated (quantitatively and qualitatively) to provide a defined and optimally balanced nutritional environment that selectively promoted the growth of a specific cell line. MCDB 105 and 110 are modifications of MCDB 104 medium, optimized for long-term survival and rapid clonal growth of human diploid fibroblast-like cells (WI-38, MRC-5, IMR-90) and low passaged human foreskin fibroblasts using FBSP, hormone, and growth factor supplements. MCDB 151, 201, and 302 are modifications of Ham's nutrient mixture F-12, designed for the growth of human keratinocytes, clonal growth of chicken embryo fibroblasts (CEF) and Chinese hamster ovary (CHO) cells using low levels of FBSP, extensive trace elements or no serum protein.

RPMI-1640 was developed by Moore et al. at Roswell Park Memorial Institute, hence the acronym RPMI. The formulation is based on the RPMI-1630 series of media utilizing a bicarbonate buffering system and alterations in the amounts of amino acids and vitamins. RPMI-1640 medium has been used for the culture of human normal and neoplastic leukocytes. RPMI-1640, when properly supplemented, has demonstrated wide applicability for supporting growth of many types of cultured cells, including fresh human lymphocytes in the 72 hour phytohemaglutinin (PHA) stimulation assay.

The culture medium can be supplemented with one or more components including, for example, serum (e.g., fetal bovine serum (FBS), preferably about 2-15% (v/v); equine (horse) serum (ES); human serum (HS)); beta-mercaptoethanol (BME), preferably about 0.001% (v/v); one or more growth factors, for example, platelet-derived growth factor (PDGF), epidermal growth factor (EGF), basic fibroblast growth factor (bFGF), insulin-like growth factor-1 (IGF-1), leukemia inhibitory factor (LIF), vascular endothelial growth factor (VEGF), and erythropoietin (EPO); amino acids, including L-valine; and one or more antibiotic and/or antimycotic agents to control microbial contamination, such as, for example, penicillin G, streptomycin sulfate, amphotericin B, gentamicin, and nystatin, either alone or in combination.

To improve proliferation of these cells, the stem cells of the invention can be co-cultured with dendritic cells or with antigen-presenting cells. These co-cultures can be carried out using basal or propagation culture conditions, as described herein. Dendritic cells can also be cultured using 10% pooled human serum (PHS) in standard culture medium plus antibiotics. We have observed that use of human serum results in the stem cells growing better (e.g., in 1%-2% PHS) as compared to bovine serum. Alternatively, serum free media may be used.

ELA stem cells can be cultured in standard tissue culture conditions, e.g., in tissue culture dishes or multiwell plates. ELA stem cells can also be cultured using a hanging drop method. In this method, ELA stem cells are suspended at about 1×10⁴ cells per mL in about 5 mL of medium, and one or more drops of the medium are placed on the inside of the lid of a tissue culture container, e.g., a 100 mL Petri dish. The drops can be, e.g., single drops, or multiple drops from, e.g., a multichannel pipetter. The lid is carefully inverted and placed on top of the bottom of the dish, which contains a volume of liquid, e.g., sterile PBS sufficient to maintain the moisture content in the dish atmosphere, and the stem cells are cultured.

In embodiments of the transplants provided herein, the cells may be cultured in the presence of an extracellular matrix. Suitable procedures for proliferating cells in the presence of such matrices are described, for example, in U.S. Pat. No. 7,297,539.

Stem cells of the invention can be cultured in a number of different ways to produce a set of lots, e.g., a set of individually-administered doses of transplants of the invention. Such lots can, for example, be obtained from stem cells of the invention from blood, bone marrow, synovial fluid or other bodily tissue. Sets of lots of stem cells of the invention can be arranged in a bank of cells for e.g., long-term storage.

Stem cells of the invention are collected, purified and suspended in an appropriate volume of culture medium and defined as Passage 0 cells. Passage 0 cells are then used to seed expansion cultures. Expansion cultures are then used to seed expansion cultures. Expansion cultures can be any arrangements of separate culture aspirates, elgl, a Cell Factory by NUNC™ Cells in the Passage 0 culture can be subdivided to any degree so as to seed expansion cultures with an inoculum of cells, e.g. about 1×10³, 2×10³, 3×10³, 4×10³, 5×10³, 6×10³, 7×10³, 8×10³, 9×10³, 10×10³, 2×10⁵, 3×10⁵, 4×10⁵, 5×10⁵, 6×10⁵, 7×10⁵, 8×10⁵, 9×10⁵, or about 10×10⁵ cells. Passage 0 cells are used to seed each expansion culture. The number of expansion cultures can depend upon the number of passage 0 cells, and may be greater or fewer in number depending upon the particular collection of cells from the fluid or tissue from the body.

Expansion cultures are grown until the density of cells in culture reaches a certain amount, e.g., 1×10⁵cells/cm². Cells can either be collected and cryopreserved at this point, or passaged into new expansion cultures as described above. Cells can be passaged, e.g., 2, 3, 4, 5, 6, 7, 8, 9, 10, 11, 12, 13, 14, 16, 18, or 20 times prior to use. A record of the cumulative number of population doublings is preferably maintained during expansion culture(s). The cells from passage 0 culture can be expanded for about 2 doublings, for example about, 3, 4, 5, 6, 7, 8, 9, 10, 11, 12, 13, 14, 16, 18, 20, 22, 24, 26, 28, 30, 32, 34, 36, 38, or about 40 doublings, or up to 60 doublings. Preferably, however, the number of population doublings, prior to dividing the population of cells into individual doses, is between about 10 and 30, preferably about 20 doublings. The cells can be cultured continuously throughout the expansion process, or can be frozen at one or more points during expansion e.g. to provide frozen preserved transplants.

Cells to be used for individual doses can be frozen, e.g., cryopreserved for later transplant use. Individual doses can comprise, e.g., about 1,000 to about 100 million cells per ml, and can comprise about 10′ and about 10⁹ cells in total.

In a specific embodiment, of the method, Passage 0 cells are cultured for a first number of doublings, e.g., approximately 4 doublings, then frozen in a first cell bank. Cells from the first cell bank are frozen and used to seed a second cell bank, the cells of which are expanded for a second number of doublings, e.g., about another eight doublings. Cells at this stage are collected and frozen and used to seed new expansion cultures that are allowed to proceed for a third number of doublings, e.g., about eight additional doublings, bringing the cumulative number of cell doublings to about 20. Cells at the intermediate points in passaging can be frozen in units of about 100,000 to about 10 million cells per ml, preferably about 1 million cells per ml for use in subsequent expansion culture. Cells at about 20 doublings can be frozen in individual doses of between about 1,000 to 100 million cells per ml for administration or use in making a stem cell-containing composition.

In one aspect, therefore, the invention provides a method of making a stem cell bank of the transplants of the invention, comprising: expanding primary culture stem cells of the invention for a first plurality of population doublings; cryopreserving said stem cells to form a Master Cell Bank; expanding a plurality of cells from the Master Cell Bank for a second plurality of population doublings; cryopreserving said cells to form a Working Cell Bank; expanding a plurality of stem cells from the Working Cell Bank for a third plurality of population doublings; and cryopreserving said stem cells in individual transplant doses, wherein said individual doses collectively compose a bank of transplants of the invention.

In another specific aspect, said first plurality of population doublings is about four population doublings; said second plurality of population doublings is about eight population doublings; and said third plurality of population doublings is about eight populations doublings.

In another specific aspect, said individual doses comprise from 10³ to about 10⁹ to about 10²³ stem cells for the transplants of the invention.

In a preferred embodiment, the donor from which the stem cells of the invention are obtained is tested for at least one pathogen. If the donor tests positive for a tested pathogen, the entire lot of cells obtained from the donor is discarded. Such testing can be performed at any time during production of the stem cell lots, including before or after establishment of Passage 0 cells, or during expansion culture. Pathogens for which the presence is tested can include, without limitation, hepatitis A, hepatitis B, hepatitis C, hepatitis D, hepatitis E, human immunodeficiency virus (types I and II), cytomegalovirus, herpesvirus, and the like.

ELA stem cell populations can be preserved as transplants, that is, placed under conditions that allow for long-term storage, or conditions that inhibit cell death by, e.g., apoptosis or necrosis. Cryoprotectants include sugars (e.g., glucose, trehalose), glycols such as glycerol (e.g., 5-20% v/v in culture media), ethylene glycol, and propylene glycol, dextran, and dimethyl sulfoxide (DMSO) (e.g., 5-15% in culture media

ELA stem cell transplants can be preserved using, e.g., a composition comprising an apoptosis inhibitor, necrosis inhibitor and/or an oxygen-carrying perfluorocarbon, as described in related U.S. provisional application No. 60/754,969, entitled “Improved Medium for Collecting Placental stem cells and Preserving Organs,” filed on Dec. 25, 2005. In one embodiment, the invention provides a method of preserving a population for transplants comprising contacting said transplants with a stem cell collection composition comprising an inhibitor of apoptosis and an oxygen-carrying perfluorocarbon, wherein said inhibitor of apoptosis is present in an amount and for a time sufficient to reduce or prevent apoptosis in the population of stem cells, as compared to a population of stem cells not contacted with the inhibitor of apoptosis. In a specific embodiment, said inhibitor of apoptosis is a caspase inhibitor. In another specific embodiment, said inhibitor of apoptosis is a JNK inhibitor. In a more specific embodiment, said JNK inhibitor does not modulate differentiation or proliferation of said stem cells. In another embodiment, said transplant collection composition comprises said inhibitor of apoptosis and said oxygen-carrying perfluorocarbon in separate phases. In another embodiment, said transplant collection composition comprises said inhibitor of apoptosis and said oxygen-carrying perfluorocarbon in an emulsion. In another embodiment, the transplant collection composition additionally comprises an emulsifier, e.g., lecithin. In another embodiment, said apoptosis inhibitor and said perfluorocarbon are between about 0 C and about 25 C at the time of contacting the stem cells. In another more specific embodiment, said apoptosis inhibitor and said perfluorocarbon are between about 2 C and 10 C, or between about 2 C and about 5 C, at the time of contacting the stem cells. In another more specific embodiment, said contacting is performed during transport of said transplant. In another more specific embodiment, said contacting is performed during freezing and thawing of said population of stem cells.

In another embodiment, the invention provides a method of preserving a transplant of an ELA stem cell population comprising contacting said transplant with an inhibitor of apoptosis and an organ-preserving compound, wherein said inhibitor of apoptosis is present in an amount and for a time sufficient to reduce or prevent apoptosis in the transplant, as compared to a transplant not contacted with the inhibitor of apoptosis. In a specific embodiment, the organ-preserving compound is UW solution (described in U.S. Pat. No. 4,798,824; also known as ViaSpan; see also Southard et al., Transplantation 49(2):251-257 (1990)) or a solution described in Stern et al., U.S. Pat. No. 5,552,267. In another embodiment, said organ-preserving compound is hydroxyethyl starch, lactobionic acid, raffinose, or a combination thereof. In another embodiment, the transplant composition additionally comprises an oxygen-carrying perfluorocarbon, either in two phases or as an emulsion.

In another embodiment of the method, the transplant is contacted with a stem cell collection composition comprising an apoptosis inhibitor and oxygen-carrying perfluorocarbon, organ-preserving compound, or combination thereof, during perfusion. In another embodiment, said stem cells are contacted during a process of tissue disruption, e.g., enzymatic digestion. In another embodiment, the ELA stem cell transplant is contacted with said stem cell collection compound after collection by perfusion, or after collection by tissue disruption, e.g., enzymatic digestion.

Typically, during ELA transplant preparation it is preferable to minimize or eliminate cell stress due to hypoxia and mechanical stress. In another embodiment of the method, therefore, a population including stem cells or a transplant is exposed to a hypoxic condition during collection, enrichment or isolation for less than six hours during said preservation, wherein a hypoxic condition is a concentration of oxygen that is less than normal blood oxygen concentration. In a more specific embodiment, said population of stem cells is exposed to said hypoxic condition for less than about two hours during said preservation. In another more specific embodiment, said population of stem cells is exposed to said hypoxic condition for less than about one hour, or less than about thirty minutes, or is not exposed to a hypoxic condition, during collection, enrichment or isolation. In another specific embodiment, said population of stem cells is not exposed to shear stress during collection, enrichment or isolation.

The ELA stem cell transplant can be cryopreserved, e.g., in cryopreservation medium in small containers, e.g., ampoules. Suitable cryopreservation medium includes, but is not limited to, culture medium including, e.g., growth medium, or cell freezing medium, for example commercially available cell freezing medium, e.g., C2695, C2639 or C6039 (Sigma). Cryopreservation medium preferably comprises DMSO (dimethylsulfoxide), at a concentration of, e.g., about 10% (v/v). Cryopreservation medium may comprise additional agents, for example, methylcellulose and/or glycerol. ELA stem cells are preferably cooled at about 1 C/min during cryopreservation. A preferred cryopreservation temperature is about −80 C. to about −180 C., preferably about −125 C. to about −140 C. Cryopreserved cells can be transferred to liquid nitrogen prior to thawing for use. In some embodiments, for example, once the ampoules have reached about −90 C., they are transferred to a liquid nitrogen storage area. Cryopreservation can also be done using a controlled-rate freezer. Cryopreserved cells preferably are thawed at a temperature of about 25 C. to about 40 C., preferably to a temperature of about 37 C.

Other preservation methods are described in U.S. patents having U.S. Pat. Nos. 5,656,498, 5,004,681, 5,192,553, 5,955,257, and 6,461,645. Methods for banking stem cells are described, for example, in U.S. patent application publication number 2003/0215942.

The production of the transplant includes cells that can be either maintained in an undifferentiated state or directed to undergo differentiation into extra-embryonic or somatic lineages ex vivo or in vivo, allows for the measuring parameters of the cellular and molecular biology of events of early human development, generation of differentiated cells from the stem cells for use in transplantation (e.g., autologous or allogenic transplantation), treating diseases (e.g., any described herein), tissue generation, tissue engineering, in vitro drug screening or drug discovery, and cryopreservation. The transplants of the invention whether autologous or allogenic can be used to treat any disease, disorder or condition that is amenable to treatment by administration of a population of differentiated or undifferentiated stem cells. As used herein, “treat” encompasses the cure of, remediation of, improvement of, lessening of the severity of or reduction in the time course of, a disease, disorder or condition or any parameter or symptom thereof.

ELA stem cell transplants can be administered in an undifferentiated state or induced to differentiate into a particular cell type, either ex vivo or in vivo, in preparation for administration to an individual in need of stem cells, or cells differentiated from stem cells. For example, ELA stem cell transplants can be injected into a damaged organ, and for organ neogenesis and repair of injury in vivo. Such injury may be due to such conditions and disorders including, but not limited to, myocardial infarction, seizure disorder, multiple sclerosis, stroke, hypotension, cardiac arrest, ischemia, inflammation, thyroiditis, age-related loss of cognitive function, radiation damage, cerebral palsy, neurodegenerative disease, Alzheimer's disease, Parkinson's disease, Leigh disease, AIDS dementia, memory loss, amyotrophic lateral sclerosis, dystrophy, ischemic renal disease, brain or spinal cord trauma, heart-lung bypass, glaucoma, retinal ischemia, or retinal trauma. ELA stem cell transplants can also be injected into localized areas in a differentiated or undifferentiated state, with or without the aid of a scaffold, matrix, oxygenated matrix, bioactive glass, bone morphogenic protein or other substance to aid in the regeneration of bone.

Transplants of the invention may also be used in promoting tissue generation, e.g., to replace damaged or diseased tissue. The term “promoting tissue generation” includes activating, enhancing, facilitating, increasing, inducing, initiating, or stimulating the growth and/or proliferation of tissue, as well as activating, enhancing, facilitating, increasing, inducing, initiating, or stimulating the differentiation, growth, and/or proliferation of tissue cells. Thus, the term includes initiation of tissue generation, as well as facilitation or enhancement of tissue generation already in progress. The term “generation” also includes the generation of new tissue and the regeneration of tissue where tissue previously existed.

Transplants of the invention have the potential to differentiate into a variety of cell types including but not limited to a neuron, chondroblast, osteoblast, adipocyte, hepatocyte, muscle cell (e.g., smooth muscle or skeletal muscle), cardiac cell, pancreatic cell, pulmonary cell, and endothelial cell. Accordingly, stem cells of the invention can be transplanted into a subject, engrafted into a target tissue, and differentiated in vivo to match the tissue type and supplement the target tissue, thereby restoring or enhancing function. In other cases, a stem cell is differentiated into a particular target tissue prior to transplantation.

The ELA stem cell transplant is capable of differentiating into neuronal tissue as evidenced into morphologic and molecular changes. Examples of the genes associated with the molecular change are OTX2, PAX6 and CAM1. Transplants of the invention or their committed differentiated progeny may also be used to treat neural disorders where regeneration or repair of tissue is desirable. Transplants of the invention can address the shortage of donor tissue for use in transplantation procedures, particularly where no alternative culture system can support growth of the required committed stem cell. In another example, following transplantation into the central nervous system (CNS), embryonic stem cell-derived neural precursors have been shown to integrate into the host tissue and, in some cases, yield functional improvement (McDonald et al., Nat. Med. 5:1410-1412, 1999).

Neurological diseases that can be treated using transplants of the invention include neurodegenerative disorders such as Parkinson's disease, polyglutamine expansion disorders (e.g., Huntington's Disease, dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer's disease, amyotrophic lateral sclerosis, ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Pelizaeus-Merzbacher disease, Pick's disease, primary lateral sclerosis, Refsum's disease, Sandhoff disease, Schilder's disease, spinal cord injury, brain injury, spinal muscular atrophy, Steele-Richardson-Olszewski disease, and Tabes dorsalis.

Neuronal differentiation of the transplant can be accomplished, for example, by placing the transplant in cell culture conditions that induce differentiation into neurons. In an example method, a neurogenic medium comprises DMEM/20% FBS and 1 mM beta-mercaptoethanol; such medium can be replaced after culture for about 24 hours with medium consisting of DMEM and 1-10 mM betamercaptoethanol. In another embodiment, the transplant is contacted with DMEM/2% DMSO/200 μM butylated hydroxyanisole. In a specific embodiment, the differentiation medium comprises serum-free DMEMIF-12, butylated hydroxyanisole, potassium chloride, insulin, forskolin, valproic acid, and hydrocortisone. In another embodiment, neuronal differentiation is accomplished by plating transplant on laminin-coated plates in Neurobasal-A medium (Invitrogen, Carlsbad Calif.) containing B27 supplement and L-glutamine, optionally supplemented with bFGF and/or EGF. The transplants can also be induced to neural differentiation by co-culture with neural cells, or culture in neuron-conditioned medium. In another embodiment, stem cells of the invention can be induced to differentiate into neural cells using, for example, commercially available products such as NEUROCULT (Stem Cell Technologies).

Neuronal differentiation can be assessed, e.g., by detection of neuron-like morphology (e.g., bipolar cells comprising extended processes) detection of the expression of e.g., nerve growth factor receptor and neurofilament heavy chain genes by RT-PCR; or detection of electrical activity, e.g., by patch-clamp. A transplant is considered to have differentiated into neuronal cells when the cells display one or more of these characteristics.

U.S. Pat. No. 6,497,872, incorporated by reference in its entirety herein, describes the differentiation of stem cells into neural cells (e.g., neurons, astrocytes, and oligodendrocytes), and methods for neurotransplantation in the undifferentiated or differentiated state, into a subject to alleviate the symptoms of neurological disease, neurodegeneration and central nervous system (CNS) trauma. Methods for the generation of suitable in autografts, xenografts, and allografts are also described.

Adipogenic differentiation of the ELA stem cell transplants can be accomplished, for example, by placing the transplant in cell culture conditions that induce differentiation into adipocytes. A preferred adipogenic medium comprises MSCGM (Cambrex) or DMEM supplemented with 15% human serum. In one embodiment, ELA stem cells are fed Adipogenesis Induction Medium (Cambrex) and cultured for 3 days (at 37 C, 5% CO₂), followed by 1-3 days of culture in Adipogenesis Maintenance Medium (Cambrex). After 3 complete cycles of induction/maintenance, the cells are cultured for an additional 7 days in adipogenesis maintenance medium, replacing the medium every 2-3 days.

In another embodiment, cells are cultured in medium comprising 1 uM dexamethasone, 0.2 mM indomethacin, 0.01 mg/ml insulin, 0.5 mM IBMX, DMEM-high glucose, FBS, and antibiotics. The ELA stem cell transplant can also be induced towards adipogenesis by culture in medium comprising one or more glucocorticoids (e.g., dexamethasone, indomethasone, hydrocortisone, cortisone), insulin, a compound which elevates intracellular levels of cAMP (e.g., dibutyryl-cAMP; 8-CPT-cAMP (8-(4)chlorophenylthio)-adenosine, 3′, 5′ cyclic monophosphate); 8-bromo-cAMP; dioctanoyl-cAMP; forskolin) and/or a compound which inhibits degradation of cAMP (e.g., a phosphodiesterase inhibitor such as isobutylmethylxanthine (IBMX), methyl isobutylxanthine, theophylline, caffeine, indomethacin).

A hallmark of adipogenesis is the development of multiple intracytoplasmic lipid vesicles that can be easily observed using the lipophilic stain oil red 0. Expression of lipase and/or fatty acid binding protein genes is confirmed by RT/PCR in cells that have begun to differentiate into adipocytes. A cell is considered to have differentiated into an adipocytic cell when the cell displays one or more of these characteristics.

Adipocytes can also be differentiated on a solid support, as described in U.S. Pat. No. 6,709,864.

The transplant of the invention may also be used for generation of tissue engineered constructs or grafts, such as for use in replacement of bodily tissues and organs (e.g., fat, liver, smooth muscle, osteoblasts, kidney, liver, heart, and neural tissue). Transplants of the invention may also be particularly well suited for the generation of tissue engineered constructs for use in replacement of musculoskeletal tissues (e.g., cartilage, bone, joint, ligament, tendon).

For instance, the inability to use articular cartilage for self-repair is a major problem in the treatment of patients who have their joints damaged by traumatic injury or suffer from degenerative conditions, such as arthritis or osteoarthritis. New approaches to cartilage tissue repair based on implanting or injecting expanded autologous cells into a patient's injured cartilage tissue can be used. More recently, it has been proposed in EP-A-0 469 070, incorporated by reference herein in its entirety, to use a biocompatible synthetic polymeric matrix seeded with chondrocytes, fibroblasts or bone-precursor cells as an implant for cartilaginous structures. Transplants of the invention can be differentiated to contain chondroblasts, and optionally seeded on a matrix for implantation into a patient in need of cartilage replacement. Undifferentiated stem cells of the invention can also be seeded on a matrix ex vivo or implanted on a matrix in vivo for a patient in need of cartilage repair or replacement. A suitable matrix is described, for example, in U.S. Pat. No. 6,692,761, incorporated by reference in its entirety herein, in a material that has hydrogel properties and allows for diffusion through the material itself, in addition to diffusion through its porous structure. This feature is highly advantageous when cells are seeded onto the scaffold and are cultured thereon, as it enables a very efficient transport of nutrient and waste materials from and to the cells. Secondly, the material closely mimics the structure and properties of natural cartilage, which, containing 80% water, is also a hydrogel. Other matrix cell based cultures are described in U.S. Pat. Nos. 5,855,619 and 5,962,325.

Methods of transplanting stem cells, stem cell-derived progeny (e.g., differentiated cells) and/or stem cell-derived tissue grafts are well known in the art. For example, U.S. Pat. No. 7,166,277, (“the ‘277 patent”), incorporated by reference in its entirety herein, describes the use of stem cells and their progeny as neuronal tissue grafts. The methods taught in the ‘277 patent for the in vitro proliferation and differentiation of stem cells and stem cell progeny into neurons and/or glia for the treatment of neurodegenerative diseases can be applied to the stem cells of the invention. Differentiation occurs by exposing the cell population to a culture medium containing a growth factor which induces the cells to differentiate. Proliferation and/or differentiation can be done before or after transplantation, and in various combinations of in vitro or in vivo conditions, including (1) proliferation and differentiation in vitro, then transplantation, (2) proliferation in vitro, transplantation, then further proliferation and differentiation in vivo, and (3) proliferation in vitro, transplantation and differentiation in vivo. As another example, U.S. Pat. No. 7,150,990, incorporated by reference in its entirety herein, describes methods for transplanting stem cells and/or stem cell-derived hepatocytes into a subject to supplement or restore liver function in vivo. Such methods can also be applied to the transplants of the invention. As yet another example, U.S. Pat. No. 7,166,464, incorporated by reference in its entirety herein, provides methods for the formation of a tissue sheet comprised of living cells and extracellular matrix formed by the cells, whereby the tissue sheet can be removed from the culture container to generate a genetically engineered tissue graft. Practitioners can follow standard methodology known in the art to transform the stem cells of the invention into a desired cell type or engineered construct for use in transplantation.

Transplants of the invention may be used to produce muscle cells (e.g., for use in the treatment of muscular dystrophy (e.g., as Duchenne's and Becker's muscular dystrophy and denervation atrophy). See, e.g., U.S. patent application publication number 2003/0118565.

Chondrogenic differentiation of ELA stem cells can be accomplished, for example, by placing the ELA stem cells transplant in cell culture conditions that induce differentiation into chondrocytes. A preferred chondrocytic medium comprises MSCGM (Cambrex) or DMEM supplemented with 15% human serum. In one embodiment, the ELA stem cell population is aliquoted into a sterile polypropylene tube, centrifuged (e.g., at 150 ×g for 5 minutes), and washed twice in Incomplete Chondrogenesis Medium (Cambrex). The cells are resuspended in Complete Chondrogenesis Medium (Cambrex) containing 0.01 μg/ml TGF-beta-3 at a concentration of about 1-20×10⁵ cells/ml. In other embodiments, the ELA stem cells transplant is contacted with exogenous growth factors, e.g., GDF-5 or transforming growth factor beta3 (TGF-beta3), with or without ascorbate. Chondrogenic medium can be supplemented with amino acids including proline and glutamine, sodium pyruvate, dexamethasone, ascorbic acid, and insulin/transferrin/selenium. Chondrogenic medium can be supplemented with sodium hydroxide and/or collagen. The ELA transplant cells may be cultured at high or low density. Cells are preferably cultured in the absence of serum.

Chondrogenesis can be assessed by e.g., observation of production of esoinophilic ground substance, safranin-O staining for glycosaminoglycan expression; hematoxylin/eosin staining, assessing cell morphology, and/or RT/PCR confirmation of collagen 2 and collagen 9 gene expression. Chondrogenesis can also be observed by growing the stem cells in a pellet, formed, e.g., by gently centrifuging stem cells in suspension. After about 1-28 days, the pellet of stem cells begins to form a tough matrix and demonstrates a structural integrity not found in non-induced, or non-chondrogenic, cell lines, pellets of which tend to fall apart when challenged. Chondrogenesis can also be demonstrated, e.g., in such cell pellets, by staining with a stain that stains collage, e.g., Sirius Red, and/or a stain that stains glycosaminoglycans (GAGs), such as, e.g., Alcian Blue. A cell is considered to have differentiated into a chondrocytic cell when the cell displays one or more of these characteristics.

Osteogenic differentiation of ELA stem cell transplants can be accomplished, for example, by placing ELA stem cells in cell culture conditions that induce differentiation into osteocytes. Such cells of the invention may also be cultured under conditions which result in the production of bone or bone cells and related compositions. Such cells and compositions may be useful for example in treating bone diseases such as osteoporosis or to treat injuries to bone. A preferred osteocytic medium comprises MSCGM (Cambrex) or DMEM supplemented with 15% human serum, followed by Osteogenic Induction Medium (Cambrex) containing 0.1 μM dexamethasone, 0.05 mM ascorbic acid-2-phosphate, 10 mM β-glycerophosphate. In another embodiment, ELA stem cells are cultured in medium (e.g., DMEM-low glucose) containing about 10⁻⁷ to about 10⁻⁹M dexamethasone, about 10-50 μM ascorbate phosphate salt (e.g., ascorbate-2-phosphate) and about 10 nM to about 10 mM β-glycerophosphate. Osteogenic medium can also include serum, one or more antibiotic/antimycotic agents, transforming growth factor-beta (e.g., TGF-β1) and/or bone morphogenic protein (e.g., BMP-2, BMP-4, BMP-6, BMP-7 or a combination thereof).

Differentiation can be assayed using a calcium-specific stain, e.g., von Kossa staining, and RT/PCR detection of, e g , alkaline phosphatase, osteocalcin, bone sialoprotein and/or osteopontin gene expression. A cell is considered to have differentiated into an osteocytic cell when the cell displays one or more of these characteristics.

Transplants having undifferentiated stem cells or stem cells differentiated into osteocytic cells can be implanted ex vivo or in vivo and used to supplement autologous bone grafts or any allograft bone grafts or scaffolds and synthetic bone grafts or scaffolds, including but not limited to, Coreograft™, Corlok™, Duet™, Profuse™, Solo™, VG1™ ALIF, VG2™ PLIF, VG2™ Ramp, Vertigraft VG2™ TLIF, Graftech™ products, Grafton™ products, Cornerstone-SR™ Cornerstone™ Select, MD™ Series, Precision™, ‘Tangent™, Puros™, Vitoss™, Cortoss™, and Healos™. Transplants having undifferentiated ELA stem cells or ELA stem cells differentiated into osteocytic cells can also be used to supplement cellular bone matrix grafts including but not limited to Trinity™ or Trinity Evolution™. Transplants having differentiated into osteocytic cells can also be combined with bone Morphogenic proteins, including BMP2, BMP4, BMP6 and BMP7 to supplement bone formation in vivo.

Exemplary methods for forming bone are also described in U.S. Pat. No. 6,863,900, which describes enhancing bone repair by transplantation of mesenchymal stem cells. To further enhance bone formation it may be desirable to inhibit osteoclastogenesis, i.e., cells which decrease bone mass. Such methods are described in U.S. Pat. No. 6,239,157. Transplants of the invention may also be used to augment bone formation by administration in conjunction with a resorbable polymer, e.g., as described in U.S. Pat. No. 6,541,024.

Differentiation of transplants having ELA stem cells into insulin-producing pancreatic cells can be accomplished, for example, by placing the ELA stem cells or transplants in cell culture conditions that induce differentiation into pancreatic cells.

Transplants of the invention are plated onto gelatinized dishes in the presence of LIF, in expansion medium or other appropriate maintenance medium (e.g., DMEM containing 15% FBS, 1 mM sodium pyruvate, 100 U/mL penicillin, 100 μg/mL streptomycin, 2 mM glutamine, 0.1 mM Non-essential amino acids (StemCell Technologies, Catalog No. 07100), 10 ng/ml LIF, 100 μg MTG). The cells are allowed to grow for two days. Next, Differentiation Medium (15% Fetal Bovine Serum 0.1 mM MEM Non-Essential Amino Acids (StemCell Technologies, Catalog No. 07600) 2 mM L-Glutamine, and 1 mM MTG in High Glucose DMEM) is added low adherent dishes (e.g., Ultra-Low Adherent dishes, StemCell Technologies). The stem cells or transplants having stem cells are trypsinized, and resuspended in Differentiation Medium, and plated onto the low adherent plates. On the second day, the medium is exchanged for fresh Differentiation Medium. The culture continues for 4 days. Optionally, nestin positive cells are enriched. The cells are transferred to a 14 ml polystyrene tube, and allowed to settle (3-5 min). The media is removed, and replaced with ES-Cult Basal Medium-A (StemCell Technologies Catalog No. 07151) supplemented with ITS. The cells are then plated, and cultured for six days, changing media every two days. The medium is then removed, the cells are washed with PBS. Cells are then trypsinized, and the medium is replaced with Pancreatic Proliferation Medium (1× N2 Supplement-A (Catalog No. 07152), 1× B27 Supplements 50X (Catalog No. 07153), 25 ng/ml recombinant human FGF-b (Catalog No. 02634), and ES-Cult Basal Medium-A (Catalog No. 05801) to final volume of 100 ml). The cells are counted and seeded at 5×10⁵ cells/ml media in a 24 well dish. Medium is changed every 2 days for 6 days total. On the sixth day, the medium is replaced with Pancreatic Differentiation Medium (1× N2 Supplement-A, 1× B27 Supplements, 10 mM nicotinamide (Catalog No. 07154), ES-Cult™ Basal Medium-A to final volume of 100 ml). After six days, insulin production can be detected (e.g., by ELISA).

In another aspect, pancreagenic medium comprises DMEM/20% CBS, supplemented with basic fibroblast growth factor, 10 ng/ml; and transforming growth factor β-1, 2 ng/ml. This medium is combined with conditioned media from nestin-positive neuronal cell cultures at 50/50 v/v. KnockOut Serum Replacement can be used in lieu of CBS. Cells are cultured for 14-28 days, refeeding every 3-4 days.

Differentiation can be confirmed by assaying for, e.g., insulin protein production, or insulin gene expression by RT/PCR. A cell or transplant is considered to have differentiated into a pancreatic cell when the cell displays one or more of these characteristics. Other methods for pancreatic cell differentiation can be found in U.S. Pat. No. 6,022,743.

The stem cell transplants of the invention may be used in the treatment of cardiac conditions, e.g., where cardiac tissue has been damaged. Exemplary conditions include myocardial infarction, congestive heart failure, ischemic cardiomyopathy, and coronary artery disease. Such methods are described, for example, in U.S. Pat. No. 6,534,052, incorporated herein by reference in its entirety. Here, embryonic cells are introduced surgically and implanted into the infarcted area of the myocardium. After implantation, the embryonic stem cells form stable grafts and survive indefinitely within the infarcted area of the heart in the living host. In other cases, the cells are cultured under conditions that induce differentiation into cardiac tissue prior to transplantation.

Myogenic (cardiogenic) differentiation of ELA stem cell transplants can be accomplished, for example, by placing the transplant or cells in cell culture conditions that induce differentiation into cardiomyocytes. A preferred cardiomyocytic medium comprises DMEM/20% CBS supplemented with retinoic acid, 1 μM; basic fibroblast growth factor, 10 ng/ml; and transforming growth factor beta-1, 2 ng/ml; and epidermal growth factor, 100 ng/ml. KnockOut Serum Replacement (Invitrogen, Carlsbad, Calif.) may be used in lieu of CBS. Alternatively, cells or transplants are cultured in DMEM/20% CBS supplemented with 50 ng/ml Cardiotropin-1 for 24 hours. In another embodiment, ELA stem cells can be cultured 5-7 or 10-14 days in protein-free medium, then stimulated with human myocardium extract, e.g., produced by homogenizing human myocardium in 1% HEPES buffer supplemented with 1% human serum. In another embodiment, myocardiocyte differentiation is accomplished by adding basic fibroblast growth factor to the standard serum-free culture media without growth factors. Confluent ELA stem cells or the transplants are exposed to 5-azacytidine and to retinoic acid and cultured in stem cell expansion medium afterwards. Alternatively, stem cells or transplants are cultured with either of these inducers alone or a combination and then cultured in serum-free medium with FGF-2 or BMP-4. Cultures are assessed for expression of any of Gata4, Gata6, cardiac troponin-T, cardiac troponin-1, ANP, Myf6 transcription factor, desmin, myogenin, and skeletal actin.

Differentiation can be confirmed by demonstration of cardiac actin gene expression, e.g., by RT/PCR, or by visible beating of the cell. An ELA stem cell or transplant is considered to have differentiated into a cardiac cell when the cell displays one or more of these characteristics.

Endothelial cell differentiation can be conducted according to methods known in the art. Stem cells of the invention can be plated at 0.5-1.0 10⁵ cells/cm² in basal medium (described above) with 100 ng/ml of VEGF-165 for 14 days. During the differentiation course, medium can be changed every 3-4 days. Differentiation cultures can be evaluated by Q-RT-PCR for VWF, CD31/Pecam, fms-like tyrosine kinase-1 (Flt-1), fetal liver kinase-1 (Flk-1), VE-cadherin, tyrosine kinase with Ig, and EGF homology domains 1 (Tie-1) and tyrosine kinase endothelial (Tek), every 3 days until day 10. Differentiated endothelial cells are stained for CD31, VWF, VE-cadherin, and VCAM-1 and evaluated for their ability to form tubes on ECMatrix and uptake acetylated low density lipoprotein (a-LDL). Tube formation can be induced by plating the differentiated endothelial cells according to the ECM625 angiogenesis assay (Chemicon) per the manufacturer's recommendations, and a-LDL uptake was performed by using Dil-Ac-LDL staining kit (Biomedical Technologies, Stoughton, Mass.) per the manufacturer's recommendations. Briefly, stem cells or transplants can be incubated with endothelium differentiation medium containing 10 μg/mL Dil-Ac-LDL for 4 hours at 37° C. and rinsed twice by Dil-Ac-LDL free endothelium medium. LDL uptake was visualized via fluorescence microscopy.

Hepatocyte differentiation can be conducted according to methods known in the art. Hepatocyte differentiation will be achieved by plating 0.5-1.0 10⁵ cells/cm² of ELA stem cells on 2% Matrigel-coated (BD354234; BD Biosciences, San Diego) plastic chamber slides in basal medium (described above) with 100 ng/ml FGF-4 and HGF for 15 days. During the differentiation course, medium can be changed every 3 days as needed. Differentiation cultures can be evaluated by Q-RT-PCR for HNF-3, HNF-1, CK18 and CK19 albumin, and CYB2B6 every 3 days until day 12. Differentiated cells can be evaluated by immunofluorescence microscopy for albumin, CK18, and HNF-1 protein expression. To assess the function of hepatocyte-like cells, karyotyping, telomere length, and telomerase activity measurements can be performed. Karyotyping can be conducted by plating enriched cells at 500 cells per cm² 48 hours prior to harvesting, followed by 10 μl/ml colcemid incubation for about 2 to about 3 hours. After collection with 0.25% Trypsin-EDTA cells can be lysed with a hypotonic solution and fixation in alcohol. Metaphases can be analyzed after Giemsa staining. For the telomerase assay, equal numbers of enriched cells can be lysed in 1× 3-[(3-cholamidopropyl) dimethylammonio]-1-propanesulfonic acid (CHAPS) buffer for 10 minutes on ice. Debris can then be pelleted at 13,000 g for 10 minutes. Protein can be quantified by the method of Bradford. One to two of μg protein can be used in the telomere repeat amplification protocol (TRAP). The TRAP protocol, which uses an enzyme-linked immunosorbent assay (ELISA)-based detection system to determine telomerase activity, can be done according to the manufacturer's instructions (Chemicon). Positive activity is defined as OD 450-690 reading greater than about 0.2 of test samples after subtracting heat-inactivated controls.

Smooth muscle cell differentiation can be conducted according to methods known in the art. For example, stem cells of the invention can be plated into 24-well plates at 3000 or 10⁵ cells/cm² in basal medium (described above) supplemented with 10 ng/ml PDGF and 5 ng/ml TGF-β1. During the differentiation course, medium can be changed every 3-4 days as needed. Smooth muscle cell (SMC) differentiation can be evaluated by RT-PCR for calponin, SM actin, smoothelin, gata-6, and myocardin and immunofluorescence (IF) staining for calponin, SM actin, sm22, and caldesmon.

Transplants of the invention can also be differentiated into skeletal muscle tissue. In one example, 5-Azacytidine can be used to differentiate stem cells of the invention into muscle cells. Stem cells can be plated in a variety of densities of about 1-4×10⁴ cells per cm² on glass or TPX slides coated with fibronectin, Matrigel, gelatin, or collagen (Stem Cell Technologies). The cells can then be exposed to concentrations of 5-azacytidine (e.g., 1-24 μM) for about 6 to about 48 hours duration in either 2% human serum, FBS, or serum-free medium (defined as DMEM with 2 mM L-glutamine, 50 U/ml penicillin, 50 μg/ml streptomycin supplemented with 10 ng/ml platelet-derived growth factor-BB, and epidermal growth factor (Sigma-Aldrich) and ITS-plus (Fisher Scientific International). In some experiments, cells received a further 24-hour exposure to 5-azacytidine 3 days later. Following 5-azacytidine exposure, cells were maintained in serum-free medium for up to 21 days. To augment differentiation after a few days, human serum with dexamethasone and hydrocortisone, myoblast-Conditioned medium, or Galectin-1 may be added. Myogenic differentiation can be observed by morphological criteria and immunostaining for desmin and sarcomeric myosin. Myogenic conversion can be assessed by counting the number of cells positive for desmin and MF20. Pax7, MyoD, and Myogenin expression can be similarly assessed using immunocytochemical staining.

Transplants of the invention may also be used to produce bone marrow or to enhance bone marrow engraftment. Exemplary procedures are described in U.S. Pat. Nos. 5,733,542 and 5,806,529.

Transplants of the invention may also be cultured under conditions that form hematopoietic stem cells. Exemplary methods for doing so are described in U.S. patent application publication No. 2003/0153082. Briefly, cells can be cultured in the presence of hematogenic cytokines such as stem cell factor (SCF), interleukin 3 (IL-3), interleukin 6 (IL-6), granulocyte-colony-stimulating factor (G-CSF)-either alone, or in combination with bone morphogenic proteins such as BMP-2, BMP-4, or BMP-7. Typically, at least two, three, or more than three such factors are combined to create a differentiation cocktail. In one example, embryoid bodies are cultured for 10 days, and then plated in an environment containing 100-300 ng/ml of both SCF and Flt-3L, 10-50 ng/ml of IL-3, IL-6, and G-CSF, 100 ng/ml SHH, and 5-100 ng/ml BMP-4 in a medium containing 20% fetal calf serum or in serum-free medium containing albumin, transferrin and insulin. After 8 to 15 days, hematopoietic cells can be evaluated for CD45⁺and CD34⁺expression. In another example, the cytokines and BMP-4 can be added to the culture the next day after embryoid body formation, which can further enhance the proportion of CD45⁺cells after 15 to 22 days. The presence of BMP-4 can allow the user to obtain populations in which 4, 10, or more secondary CFUs form from each primary CFU, which indicate the presence of self-renewing hematopoietic progenitors.

Functional studies described herein can be carried out to characterize the committed progeny.

Human albumin concentrations can be determined using an ELISA. Concentrations of albumin can be determined by generating standard curves from known concentrations of human albumin. Peroxidase-conjugated and affinity-purified anti-human albumin and reference human albumin can be obtained from Brigham and Women's Hospital Laboratory (Boston, Mass.). To verify specificity of results, conditioned medium from endothelial differentiations and unconditioned hepatocyte differentiation medium can be used.

Urea secretion can be assessed by colorimetric assay (DIUR-500 BioAssay Systems) per the manufacturer's instructions. Conditioned medium from endothelial differentiations and unconditioned hepatocyte differentiation medium can be used as negative controls.

Slides can be oxidized for 5 minutes in 1% periodic acid-Schiff (PAS) (Sigma-Aldrich) and rinsed several times with double-distilled H2O (ddH2O). Samples can be incubated with Schiff's reagent for 15 minutes, rinsed several times with ddH2O, immediately counterstained with hematoxylin for 1 minute, and washed several times with ddH20. The observations made in this example demonstrate that the adult synovial fluid contains a sub-population of stem cells and that with the appropriate stimuli, these cells can function as mesodermal, ectodermal, or endodermal cell types.

Transplants of the invention may also be cultured under conditions which form dendritic cells. Such cells may be useful in vaccinations against cancer by genetically altering the cells to express a cancer antigen such as telomerase reverse transcriptase (TERT). The vaccine may then be administered to a subject having a cancer or at increased risk of developing such a cancer. Exemplary differentiation procedures are described in U.S. patent application publication number 2006/0063255. Thus, differentiation can be initiated in a non-specific manner by forming embryoid bodies or culturing with one or more non-specific differentiation factors. Embryoid bodies (EBs) can be made in suspension culture. Undifferentiated stem cells can be harvested by brief collagenase digestion, dissociated into clusters or strips of cells, and passaged to non-adherent cell culture plates. The aggregates can be fed every few days, and then harvested after a suitable period, typically 4-8 days. Specific recipes for making EB cells from stem cells of are found in U.S. Pat. No. 6,602,711, International patent application WO 01/51616, and U.S. patent application publication No. 2003/0175954 and 2003/0153082. Alternatively, fairly uniform populations of more mature cells can be generated on a solid substrate; see, e.g., U.S. patent application publication No. 2002/019046.

In one example, the cells can be first differentiated into an intermediate cell (either as an isolated cell type or in situ) that has features of multipotent hematopoietic precursor cells (e.g., CD34⁺CD45^{±}CD38⁻and the ability to form colonies in a classic CFU assay). This can be accomplished by culturing with hematopoietic factors such as interleukin 3 (IL-3), BMP-4, optionally in combination with factors such SCF, Flt-3L, G-CSF, other bone morphogenic factors, or monocyte conditioned medium. The medium used can be any compatible medium (e.g., X-VIVO™ 15 expansion medium (Biowhittaker/Cambrex), and Aim V (Invitrogen/Gibco). See also WO 98/30679 and U.S. Pat. No. 5,405,772. In addition or as a substitute for some of these factors, hematopoietic differentiation can be promoted by co-culturing with a stromal cell lineage (e.g., mouse lines OP9 or Ac-6, commercially available human mesenchymal stem cells, or the hES derived mesenchymal cell line HEF1; U.S. Pat. No. 6,642,048), or by culturing medium preconditioned in stromal cells culture.

The hematopoietic intermediate can be further differentiated into antigen presenting cells or dendritic cells that may have one or more of the following features in any combination: CD40⁺, CD80⁺, CD83⁺, CD86⁺, Class II MHC⁺, highly Class I MHC⁺, CD14⁻, CCR5⁺, and CCR7⁺. This can be accomplished by culturing with factors such as GM-CSF, IL-4, or IL-13, a pro-inflammatory cytokine such as TNFα or IL-6, and interferon gamma (IFNγ).

Another approach directs transplants towards the phagocytic or dendritic cell subset early on. Intermediate cells may already bear hallmarks of monocytes ontologically related to dendritic cells or phagocytic antigen presenting cells, and may have markers such as cell surface F4/80 and Dec205, or secreted IL-12. IL-3 and/or stromal cell conditioned medium are used as before, and GM-CSF is present in the culture concurrently.

Maturation of the phagocytic or dendritic cell precursor is achieved in a subsequent step: potentially withdrawing the IL-3, but maintaining the GM-CSF, and adding IL-4 (or IL-13) and a pro-inflammatory cytokine. Other factors that may be use include IL-1β, IFNγ, prostaglandins (e.g., PGE2), and transforming growth factor beta (TGFI3); along with TNFα and/or IL-6. A more mature population of dendritic cells is thereby produced.

In either the above methods, it may be beneficial to mature the cells further by culturing with a ligand or antibody that is a CD40 agonist (U.S. Pat Nos. 6,171,795 and 6,284,742), or a ligand for a Toll-like receptor (such as LPS, a TLR4 ligand; poly I:C, a synthetic analog of double stranded RNA, which is a ligand for TLR3; Loxoribine, which a ligand for TLR7; or CpG oligonucleotides, synthetic oligonucleotides that contain unmethylated CpG dinucleotides in motif contexts, which are ligands for TLR9), either as a separate step (shown by the open arrows), or concurrently with other maturation factors (e.g., TNFα and/or IL-6).

In some embodiments, the cells are divided into two populations: one of which is used to form mature dendritic cells that are immunostimulatory, and the other of which is used to form toleragenic dendritic cells. The toleragenic cells may be relatively immature cells that are CD80⁻, CD86⁻, and/or ICAM-1⁻. They may also be adapted to enhance their toleragenic properties (e.g., transfected to express Fas ligand, or inactivated by irradiation or treatment with mitomycin c).

Transplants of the invention may also be used to inhibit or reduce undesired or inappropriate immune responses. For example, the stem cell transplants may be used to treat an autoimmune disease, to promote wound healing, or to reduce or prevent rejection of a tissue or organ. Stem cell transplants can be used to suppress immune responses upon administration to subjects. See, e.g., U.S. patent application publication number 2005/0282272. Such approaches have also been proposed in Sykes et al., Nature 435:620-627, 2005 and Passweg et al., Semin. Hematol. 44:278-85, 2007. Other immunosuppressive uses of stem cells are described in U.S. Pat. Nos. 6,328,960, 6,368,636, 6,685,936, 6,797,269, 6,875,430, and 7,029,666.

Thus, transplants of the invention may be used for immunosuppression or to treat autoimmune disease. Immunosuppression may be desirable prior to transplantation of tissues or organs into a patient (e.g., those described herein). Autoimmune disease which may be treated by administration of stem cells include multiple sclerosis (MS), systemic sclerosis (SSc), systemic lupus erythematosus (SLE), rheumatoid arthritis (RA), juvenile idiopathic arthritis, and inunune cytopenias. Other autoimmune disease which may be treated using stem cells of the include acute disseminated encephalomyelitis (ADEM), Addison's disease, Ankylosing spondylitis, antiphospholipid antibody syndrome (APS), aplastic anemia, autoimmune hepatitis, autoimmune oophoritis, celiac disease, Crohn's disease, diabetes mellitus type 1, gestational pemphigoid, Goodpasture's syndrome, Graves' disease, Guillain-Barre syndrome (GBS), Hashimoto's disease, idiopathic thrombocytopenic purpura, Kawasaki's disease, lupus erythematosus, mixed connective tissue disease, multiple sclerosis, myasthenia gravis, opsoclonus myoclonus syndrome, Ord's thyroiditis, pemphigus, pernicious anaemia, primary biliary cirrhosis, rheumatoid arthritis, Reiter's syndrome, Sjogren's syndrome, Takayasu's arteritis, temporal arteritis, warm autoimmune hemolytic anemia, and Wegener's granulomatosis.

In other embodiments, the transplants of the invention can be used to reduce or prevent rejection of a transplanted tissue or organ. For instance, such a method can include engrafting the hematopoietic system of the tissue or organ recipient with stem cells of the invention obtained from the organ donor prior to transplanting the organ. By engrafting the hematopoietic system of the recipient with stem cells derived from the organ donor, rejection of the transplanted organ is thereby inhibited. Prior to engraftment and organ transplantation, the bone marrow of the recipient would be ablated by standard methods well known in the art. Generally, bone marrow ablation is accomplished by X-irradiating the animal to be transplanted, administering drugs such as cyclophosphamide or by a combination of X-radiation and drug administration. Bone marrow ablation can be produced by administration of radioisotopes known to kill metastatic bone cells such as, for example, radioactive strontium, ¹³⁵Samarium, or ¹⁶⁶Holmium (Applebaum et al., 1992, Blood 80:1608-1613).

In some embodiments, autologous transplants can be introduced into a subject. A population of ELA stem cells can be isolated from the recipient according to the methods described herein e.g. from synovial fluid, or prior to ablating bone marrow of the recipient. The bone marrow of the individual is purged of malignant blasts and other malignant cells such that by transplanting the non-malignant stem cells back into to the individual, diseases such as melanomas may be treated.

In certain embodiments, it may be desirable to treat the cells in order to decrease the likelihood of transplant rejection, especially where non-autologous cells are used. The invention therefore features methods of decreasing uric acid production in cells, and cells in which uric acid production has been reduced. Exemplary means for doing so are described in U.S. patent application publication No. 2005/0142121 and include treatment with compounds that decrease xanthine oxidase activity, such as allopurinol, oxypurinol, and BOF-4272. Other approaches include pre-treatment with low levels of tungsten to deplete molybdenum, a necessary cofactor for xanthine oxidase. Genetic or RNAi approaches which reduce transcription or translation of the xanthine oxidase gene or mRNA, may also be used to decrease uric acid production.

Inflammation during healing of wounds has been shown to increase scarring at wound sites (Redd et al., Philos. Trans. R. Soc. Lond. B Biol. Sci. 359:777-784, 2004). The transplants of the invention can also be used to improve wound healing. Doing so at a wound site can promote healing of the tissue and further can decrease fibrosis and scarring at the wound site. Because formation of age-related wrinkles may also be caused by a scarring process, administration of stem cells of the invention to the site of wrinkles may reduce wrinkle formation or result in reduction or elimination of such of wrinkles as well as scars. Wound healing using regenerative cells from adipose tissue is described, for example, in U.S. patent application publication No. 2005/0048034 and 2006/0147430. Such approaches can be adapted for use with the cells of the present invention.

ELA stem cell transplants of the invention may be used in the production of tissues according to methods known in the art. U.S. Pat. No. 5,834,312, incorporated by reference in its entirety herein, for example, describes media and methods for the in vitro formation of a histologically complete human epithelium. The media are serum-free, companion cell or feeder layer free and organotypic, matrix free solutions for the isolation and cultivation of clonally competent basal epithelial cells. The media and methods of the invention are useful in the production of epithelial tissues such as epidermis, cornea, gingiva, and ureter. U.S. Pat. No. 5,912,175, incorporated by reference in its entirety herein, describes media and methods for the in vitro formation of human cornea and gingival from stem cells.

ELA stem cell transplants can be used to treat autoimmune conditions such as juvenile diabetes, lupus, muscular dystrophy, rheumatoid arthritis, and the like. The stem cell transplants of the invention can also be used to increase vascularization. Doing so may be desirable when organs have been injured or in cases of diabetic disorders. Diseases in which increased vascularization is desirable include diabetes, atherosclerosis, arteriosclerosis, and any of the cardiac conditions described above.

ELA stem cell transplants can be used, in specific embodiments, in autologous or heterologous enzyme replacement therapy to treat specific diseases or conditions, including, but not limited to lysosomal storage diseases, such as Tay-Sachs, Niemann-Pick, Fabry's, Gaucher's disease (e.g., glucocerbrosidase deficiency), Hunter's, and Hurler's syndromes, Maroteaux-Lamy syndrome, fucosidosis (fucosidase deficiency), Batten disease (CLN3), as well as other gangliosidoses, mucopolysaccharidoses, and glycogenoses.

ELA stem cell transplants alone or in combination with stem or progenitor cell populations, may be used alone, or as autologous or heterologous or allogeneic transgene carriers in gene therapy, to correct inborn errors of metabolism, cystic fibrosis, adrenoleukodystrophy (e.g., co-A ligase deficiency), metachromatic leukodystrophy (arylsulfatase A deficiency) (e.g., symptomatic, or presymptomatic late infantile or juvenile forms), globoid cell leukodystrophy (Krabbe's disease; galactocerebrosidase deficiency), acid lipase deficiency (Wolman disease), glycogen storage disease, hypothyroidism, anemia (e.g., aplastic anemia, sickle cell anemia, etc.), Pearson syndrome, Pompe's disease, phenylketonuria (PKU), porphyrias, maple syrup urine disease, homocystinuria, mucopolysaccharidenosis, chronic granulomatous disease and tyrosinemia and Tay-Sachs disease or treat cancer (e.g., a hematologic malignancy), tumors or other pathological conditions. The ELA stem cell transplants can be used to treat skeletal dysplasia. In one embodiment, ELA stem cell transplants transformed to express tissue plasminogen activator (tPA) and are administered to an individual to treat thrombus.

In other embodiments, stem cells may be used in autologous, allogeneic or heterologous tissue regeneration or replacement therapies or protocols, including, but not limited to treatment of corneal epithelial defects, treatment of osteogenesis imperfecta, cartilage repair, bone regeneration, facial dermabrasion, mucosal membranes, tympanic membranes, intestinal linings, neurological structures (e.g., retina, auditory neurons in basilar membrane, olfactory neurons in olfactory epithelium), burn and wound repair for injuries of the skin, or for reconstruction of other damaged or diseased organs or tissues.

In an embodiment, an ELA stem cell transplant is used in hematopoietic reconstitution in an individual that has suffered a partial or total loss of hematopoietic stem cells, e.g., individuals exposed to lethal or sub-lethal doses of radiation (whether industrial, medical or military); individuals that have undergone myeloablation as part of, e.g., cancer therapy, and the like, in the treatment of, e.g., a hematologic malignancy. ELA stem cell transplants can be used in hematopoietic reconstitution in individuals having anemia (e.g., aplastic anemia, sickle cell anemia, etc.). Preferably, the ELA stem cell transplants are administered to such individuals with a population of hematopoietic stem cells. ELA stem cell transplants can be used in place of, or to supplement, bone marrow or populations of stem cells derived from bone marrow. Typically, approximately 1×10⁸ to 2×10⁸ bone marrow mononuclear cells per kilogram of patient weight are infused for engraftment in a bone marrow transplantation (i.e., about 70 ml of marrow for a 70 kg donor). To obtain 70 ml an intensive donation and significant loss of donor blood in the donation process is used. An isolated population of ELA stem cells for hematopoietic reconstitution can comprise, in various embodiments, about at least, or no more than 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 1×10¹¹ or more stem cells.

In one embodiment, therefore, ELA stem cell transplants can be used to treat patients having a blood cancer, such as a lymphoma, leukemia (such as chronic or acute myelogenous leukemia, acute lymphocytic leukemia, Hodgkin's disease, etc.), myelodysplasia, myelodysplastic syndrome, and the like. In another embodiment, the disease, disorder or condition is chronic granulomatous disease.

Because hematopoietic reconstitution can be used in the treatment of anemias, the present invention further encompasses the treatment of an individual with a stem cell combination of the invention, wherein the individual has an anemia or disorder of the blood hemoglobin. The anemia or disorder may be natural (e.g., caused by genetics or disease), or may be artificially-induced (e.g., by accidental or deliberate poisoning, chemotherapy, and the like). In another embodiment, the disease or disorder is a marrow failure syndrome (e.g., aplastic anemia, Kostmann syndrome, Diamond-Blackfan anemia, amegakaryocytic thrombocytopenia, and the like), a bone marrow disorder or a hematopoietic disease or disorder.

ELA stem cell transplants can also be used to treat severe combined immunodeficiency disease, including, but not limited to, combined immunodeficiency disease (e.g., Wiskott-Aldrich syndrome, severe DiGeorge syndrome, and the like).

ELA stem cell transplants of the invention, alone or in combination with other stem cell or progenitor cell populations, can be used in the manufacture of a tissue or organ in vivo. The methods of the invention encompass using ELA stem cell transplants or populations of ELA stem cells, to seed a matrix and to be cultured under the appropriate conditions to allow the cells to differentiate and populate the matrix. The tissues and organs obtained by the methods of the invention can be used for a variety of purposes, including research and therapeutic purposes. In another embodiment, the methods of the invention encompass using ELA stem cell transplants or populations of ELA stem cells, to seed a matrix to allow the cells to differentiate and populate the matrix in vivo to repair damaged tissues.

In an embodiment of the invention, ELA stem cell populations and transplants may be used for autologous or allogenic transplants or transplants comprising a combination of autologous and allogeneic stem cells, including matched and mismatched HLA type hematopoietic transplants. In one embodiment of the use of ELA stem cell transplants as allogenic hematopoietic transplants, the host is treated to reduce immunological rejection of the donor cells, or to create immunotolerance (see, e.g., U.S. Pat. No. 5,800,539 and 5,806,529). In another embodiment, the host is not treated to reduce immunological rejection or to create immunotolerance.

ELA stem cell transplants, either alone or in combination with one or more other stem cell populations, can be used in therapeutic transplantation protocols, e.g., to augment or replace stem or progenitor cells of the liver, pancreas, kidney, lung, nervous system, muscular system, bone, bone marrow, thymus, spleen, mucosal tissue, gonads, or hair. Additionally, ELA stem cell transplants may be used instead of specific classes of progenitor cells (e.g., chondrocytes, hepatocytes, hematopoietic cells, pancreatic parenchymal cells, neuroblasts, muscle progenitor cells, etc.) in therapeutic or research protocols in which progenitor cells would typically be used.

In one embodiment, the invention provides for the use of ELA stem cell transplants as an adjunct to hair replacement therapy. For example, in one embodiment, ELA stem cell transplant is injected subcutaneously or intradermally at a site in which hair growth or regrowth is desired. The number of stem cells injected can be, e.g., between about 100 and about 100,000 per injection, in a volume of about 0.1 to about 1.0 μL, though more or fewer cells in a greater or lesser volume can also be used. Administration of an ELA stem cell transplant to facilitate hair regrowth can comprise a single injection or multiple injections in, e.g., a regular or a random pattern in an area in which hair regrowth is desired. Known hair regrowth therapies can be used in conjunction with the ELA stem cell transplants, e.g., topical minoxidil. Hair loss that can be treated using ELA stem cell transplants can be naturally-occurring (e.g., male pattern baldness) or induced (e.g., resulting from toxic chemical exposure).

ELA stem cell transplants of the invention can be used for augmentation, repair or replacement of cartilage, tendon, or ligaments. For example, in certain embodiments, prostheses (e.g., hip prostheses) can be coated with replacement cartilage tissue constructs grown from ELA stem cell transplants of the invention. In other embodiments, joints (e.g., knee) can be reconstructed with cartilage tissue constructs grown from ELA stem cell transplants. Cartilage tissue constructs can also be employed in major reconstructive surgery for different types of joints (see, e.g., Resnick & Niwayama, eds., 1988, Diagnosis of Bone and Joint Disorders, 2d ed., W. B. Saunders Co.). In another embodiment, ELA stem cell transplants can be seeded in a scaffold (e.g., silk and collagen) prior to or after insertion at the site of injury or disease.

ELA stem cell transplants of the invention can be used for augmentation, repair or replacement of bone. For example, in certain embodiments, prostheses (e.g., hip prostheses) can be coated with ELA stem cell transplants or tissue constructs grown from ELA stem cell transplants of the invention. In other embodiments, ELA stem cell transplants can supplement bone growth by being combined ex vivo or in vivo with autologous bone graft or any allograft bone grafts or scaffolds and synthetic bone grafts or scaffolds, including but not limited to, Coreograft™, Corlok™, Duet™, Profuse™, Solo™, VG1™ ALIF, VG2™ PLIF, VG2™ Ramp, Vertigraft VG2™ TLIF, Graftech™ products, Grafton™ products, Cornerstone-SR™, Cornerstone™ Select, MD™ Series, Precision™, Tangent™, Puros™, Vitoss™, Cortoss™, and Healos™. Undifferentiated ELA stem cell transplants or ELA stem cells differentiated into osteocytic cells can also be used to supplement cellular bone matrix grafts including but not limited to Trinity or Trinity Evolution™. Undifferentiated ELA stem cell transplants or ELA stem cell transplants differentiated into osteocytic cells can also be combined with bone Morphogenic proteins, including BMP2, BMP4, BMP6 and BMP7(e.g. Infuse or OP-1) to supplement bone formation in vivo. Differentiated or undifferentiated expanded or non-expanded ELA stem cell transplants can be introduced into the subject by localized injection or systemic injection.

The ELA stem cell transplants of the invention can be used to repair damage to tissues and organs resulting from, e.g., trauma, metabolic disorders, or disease. The trauma can be, e.g., trauma from surgery, e.g., cosmetic surgery. In such an embodiment, a patient can be administered an ELA stem cell transplant alone or combined with other stem or progenitor cell populations, to regenerate or restore tissues or organs which have been damaged as a consequence of disease or injury.

The invention provides a method to generate bone within an organism. Generally, the method involves implanting a mammalian ELA stem cell transplant into an organism. Preferably the mammal is a human. More preferably the organism is a human. The human ELA cells may be obtained from one human and implanted into the same or different human. The human ELA cells may be used as a transplant in an unexpanded state or the transplant may be ex vivo expanded prior to being implanted into the organism. Preferably the human ELA cell transplant is non-induced prior to being implanted into the organism or the human ELA cell transplant is induced with BMP-4 or mineralizing induction prior to being implanted. A human postnatal ELA stem cell that is not in combination with a carrier can be implanted into an organism. A human ELA cell transplant that is in combination with a carrier can be implanted into an organism. Preferably, the carrier contains hydroxyapatite or tricalcium phosphate, or a combination of both. The human ELA cell transplant may induce a recipient cell to differentiate into bone-forming cells. The method of the invention can be used to promote bone formation at a site of trauma within an organism. The trauma may or may not be produced by a physical injury. For example, the physical injury is an accidental physical injury. Alternatively, the physical injury results from a medical or dental procedure, for example, from surgery. The trauma may be due to degenerative disease, for example, osteoporosis.

The invention provides a method of using the ELA stem cell transplant and performing an in utero transplantation of a population of the cells to form chimerism of cells or tissues, thereby producing human cells in prenatal or post-natal humans or animals following transplantation, wherein the cells produce therapeutic enzymes, proteins, or other products in the human or animal so that genetic defects are corrected.

The invention provides a method for inducing an immune response to an infectious agent in a human subject involving genetically altering an expanded clonal population of an ELA stem cells transplant in culture to express one or more pre-selected antigenic molecules that elicit a protective immune response against an infectious agent, and introducing into the subject an amount of the genetically altered cells which effectively induce the immune response. The present method may further involve, prior to the second step, the step of differentiating the ELA stem cells or transplant to form dendritic cells.

The present invention provides a method of using ELA stem cell transplants to identify genetic polymorphisms associated with physiologic abnormalities, involving obtaining the ELA stem cell from a statistically significant population of individuals from whom phenotypic data can be obtained, optionally culture expanding the ELA stem cells from the statistically significant population of individuals to establish ELA stem cell cultures, identifying at least one genetic polymorphism in the cultured ELA stem cell population, inducing the cultured ELA stem cells to differentiate, and characterizing aberrant metabolic processes associated with at least one genetic polymorphism by comparing the differentiation pattern exhibited by an ELA stem cell having a normal genotype with the differentiation pattern exhibited by an ELA stem cell having an identified genetic polymorphism. The method is carried out on each individual separately, for example each in a well of a multi-well culture plate, or using sibling pools of about 5, 10, 50 or about 100 individuals together in each well.

The present invention also provides a method for treating cancer in a mammalian subject involving preparing genetically altered ELA stem cell transplants that express a tumoricidal protein, an anti-angiogenic protein, or a protein that is expressed on the surface of a tumor cell in conjunction with a protein associated with stimulation of an immune response to antigen, and introducing an effective anti-cancer amount of the genetically altered ELA stem cell transplant into the mammalian subject.

This invention provides methods for alleviating chronic pain and/or spasticity by administering a transplant having a cell population including ELA stem cells to thereby treat chronic pain and/or spasticity. Preferably, such treatment results in reestablishing sensory neural pathways in the subject with chronic pain. The present invention is based, at least in part, on the discovery that neural cell populations can be administered into the spinal cord (e.g., to the subarachnoid space or to the spinal dorsal horn) of a subject to treat chronic pain and/or spasticity. ELA stem cell transplants can also be used to suppress inflammatory cytokines which induce pain. A method for treating TNF mediated dementias, including Alzheimer's Disease, Pick's Disease, Lewy Body Disease and Idiopathic Dementia, in a human by inhibiting the action of tumor necrosis factor (TNF) through the administration of an ELA stem cells transplant by administering said dose parenterally by perispinal administration into the perispinal space without direct intrathecal injection.

The present invention provides transplants comprising ELA stem cells, or biomolecules therefrom. The ELA stem cell transplants of the present invention can be combined with any physiologically-acceptable (e.g. platelets or bone morphogenic protein) or medically-acceptable compound, composition or device for use in, e.g., research or therapeutics.

The ELA stem cell transplants of the invention can be preserved, for example, cryopreserved for later use. Methods for cryopreservation of cells, such as stem cells, are well known in the art. ELA stem cell transplants can be prepared in a form that is easily administrable to an individual. For example, the invention provides an ELA stem cell transplant that is contained within a container that is suitable for medical use. Such a container can be, for example, a sterile plastic bag, flask, jar, or other container from which the ELA stem cell transplant can be easily dispensed. For example, the container can be a blood bag or other plastic, medically-acceptable bag, syringe or vial suitable for the localized or intravenous administration of a liquid to a recipient. The container is preferably one that allows for cryopreservation of the combined stem cell population.

The cryopreserved ELA stem cell transplant can comprise ELA stem cells derived from a single donor, or from multiple donors. The ELA stem cell transplant can be HLA-matched to an intended recipient, or partially matched or completely HLA-unmatched.

Thus, in one embodiment, the invention provides a transplant comprising an ELA stem cell in a container. In a specific embodiment, the ELA stem cell transplant is cryopreserved. In another specific embodiment, the container is a bag, flask, syringe, vial or jar. In more specific embodiment, said bag is a sterile plastic bag. In a more specific embodiment, said bag is suitable for, allows or facilitates intravenous administration of said transplant. The bag can comprise multiple lumens or compartments that are interconnected to allow mixing of the transplant and one or more other solutions, e.g., a drug, prior to, or during, administration. In another specific embodiment, the container is a vial or syringe that is suitable for, allows or facilitates administration of said transplant into a site of injury, wound, scaffold, or other localized area. In another specific embodiment, the transplant comprises one or more compounds that facilitate cryopreservation of the combined stem cell population. In another specific embodiment, said transplant is contained within a physiologically-acceptable aqueous solution. In a more specific embodiment, said physiologically-acceptable aqueous solution is a 0.9% NaC1 solution. In another specific embodiment, said transplant comprises ELA stem cells that are HLA-matched to a recipient of said stem cell population. In another specific embodiment, said combined ELA stem cell transplant comprises ELA stem cells that are at least partially HLA-mismatched to a recipient of said stem cell population. In another specific embodiment, said ELA stem cell transplant is derived from a non-related donor. In another specific embodiment, said ELA transplant contains cells derived from a plurality of donors.

Other preservation methods are described in U.S. Pat. Nos. 5,656,498, 5,004,681, 5,192,553, 5,955,257, and 6,461,645. Methods for banking stem cells are described, for example, in U.S. patent application publication No. 2003/0215942.

It is to be understood, however, that the scope of the present invention is not to be limited to the specific embodiments described above. The invention may be practiced other than as particularly described and still be within the scope of the accompanying claims.

It should be understood that the methods described herein may be carried out in a number of ways that are well known in the art, with numerous modifications and variations thereof, such equivalents are considered to be within the scope of the invention as described herein. For example, the ELA stem cell transplant described herein is suitable for repair of cardiac muscle and endothelial tissue, for example in connection with repair of ischemic defects or physical trauma to the cardiovascular system; or suitable for use in the repair of soft tissues as described above and in the current medical literature. In particular, but without limitation ELA transplants are suitable for interchange with known MAPC and MSC based transplant products and their applications, given similar dosages based on cell count and through similar delivery techniques used with MAPC and MSC based therapies (see U.S. patent application publication No.: 2007/0134215 A1; 2006/0008450 A1; 2006/0263337 A1; 2006/0182712 A1; 2007/0059823 A1; 2007/0274970 A1; and U.S. Pat. Nos. 5,811,094; 6,368,636; 6,255,119; 5,908,784).

It may also be appreciated that any theories set forth as to modes of action or interactions between cell types should not be construed as limiting this invention in any manner, but are presented such that the methods of the invention can be more fully understood.

The claimed invention reflects contributions of the named inventors to joint research under an agreement between Parcell Laboratories, LLC and The Brigham & Women's Hospital, Inc.

The following examples and claims are exemplary and further illustrate aspects of the present invention. However, they are in no way a limitation of the teachings or disclosure of the present invention as set forth herein.

### EXAMPLES

### Example 1

### Proliferative Capacity of ELA Cells

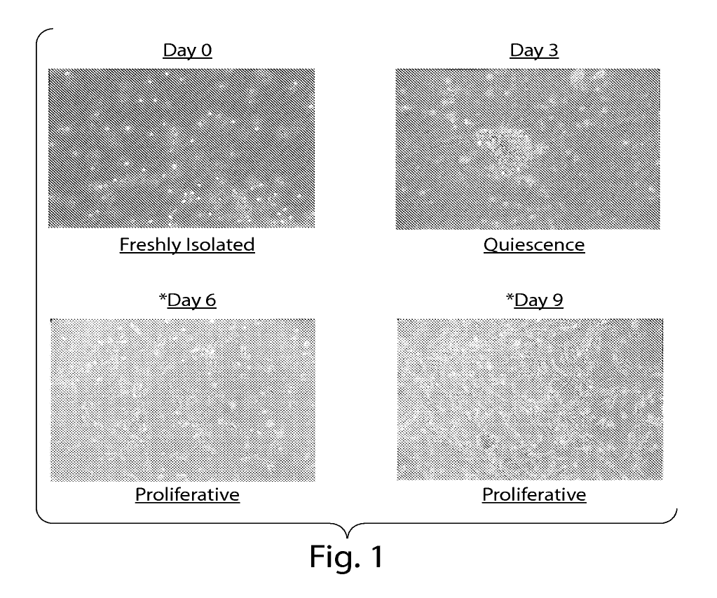

Human ELA stem cells were obtained and cultures were observed as a function of time. It was observed that cells remained quiescent at day 3. By day 6 outgrowth of cell processes indicated the start of proliferation. Further proliferation was observed at day 9 and growth continued in stages of development. An ELA stem cell colony/embryoid body at days 0, 3, 6, and 9 of primary culture is composed of uniformly spindle-shaped cells. FIG. 1 shows spindle-shaped cells that continue to populate the tissue culture surface until full confluence was established.

### Example 2

### Differentiation Capacity of ELA Cells is Multiplied

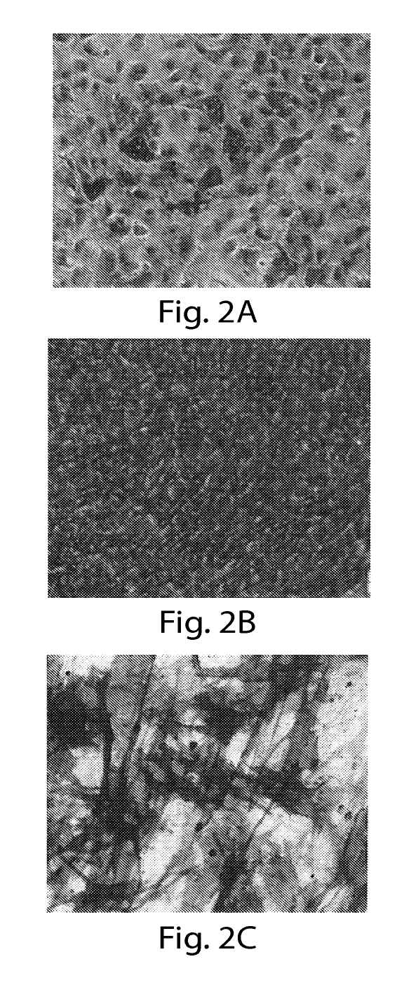

Human ELA™ stem cells were observed to differentiate into three types of mesodermal tissues. The ELA stem cells from the same donor were cultured for 21 days in adipogenic medium (FIG. 2A. Adipogenesis was demonstrated by the accumulation of neutral lipid vacuoles.), chondrogenic medium (FIG. 2B. Chondrogenesis was demonstrated by the presence of mucopolysaccharides and glycosaminoglycans of cartilage.) and osteogenic medium (FIG. 2C. Osteogenesis was demonstrated by an increase in calcium deposition.). Characteristic cytologies were observed for differentiating cultures. Cells in FIG. 2A formed an adipose tissue, as a result of culture in adiogenic medium. FIG. 2B shows cell growth typical of chondrocytes, and FIG. 2C shows a culture typical of osteocytes. In each case the proliferating ELA cells donated an extracellular matrix that was characteristic of the culture medium. These data show the potential for differentiation of the ELA cells for production of transplants.

### Example 3

### Differentiation of ELA Cells on a Matrix

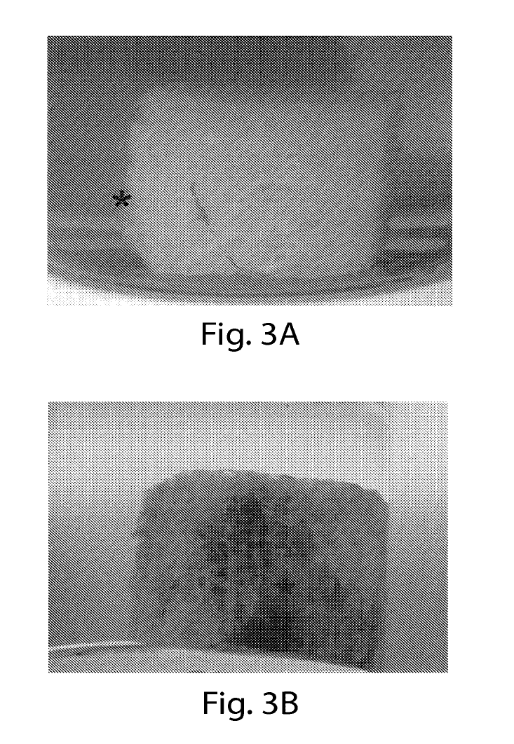

Photomicrographs of an ELA stem cell-loaded demineralized bone matrix implant show ELA stem cells loaded into demineralized bone matrix and cultured 21 days in the absence (FIG. 3A) or presence (FIG. 3B) of osteogenic media. Cultures with high levels of bone formation were stained black by alkaline phosphatase (APase). The ELA cells differentiated in the context of the matrix, and the differentiation was specific to the presence of osteogenic medium.

### Example 4

### Suppression of Pan-Activated T cells with ELA Stem Cells.

T cells express cell surface antigens that when cross-linked with antibodies produce an extremely vigorous T cell proliferative response. When treated with alloreactive ELA cells, T cell proliferation subsides.

1×10⁵T cells from an individual were cultured in u-bottom microliter wells with varying concentrations of allogeneic ELA cells or mesenchymal stem cells (MSCs). The ELA stem cells and mesenchymal stem cells (MSCs) were seeded at each of amounts 10,000, 20,000, 35,000, and 50,000, respectively. After seven days in culture, microliter wells were pulsed with ³H-thymidine for the last 18 hours of the culture period to measure T cell proliferation.

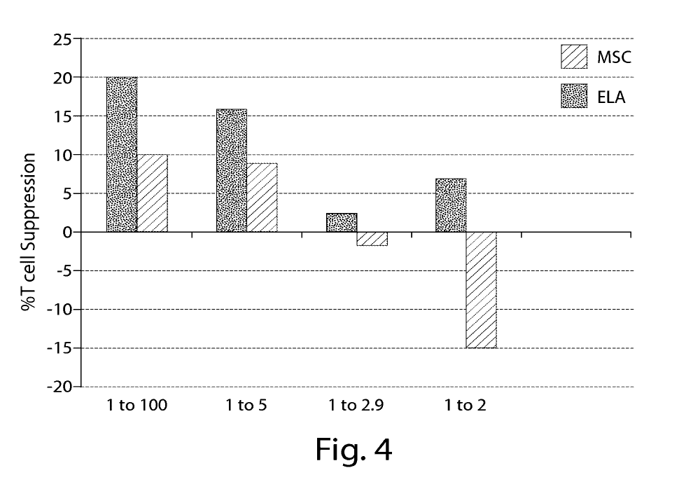

The results shown in FIG. 4 demonstrate that ELA cells and MSC suppressed pan T cell activation, at low concentrations and that the ELA cells were more efficient at T cell suppression than the MSCs. Furthermore, as the concentration of MSCs increased in the culture, the level of suppression decreased to a point where T cell proliferation increased. This finding was not observed in the ELA cell cultures with high cell numbers.

Accordingly, the ability of an ELA stem cell population to inhibit T cell activation and proliferation confirms their ability to engraft as a tissue transplant without inducing cell mediated immune responses.

### Example 5

### Suppression of NK Cell Cytotoxicity

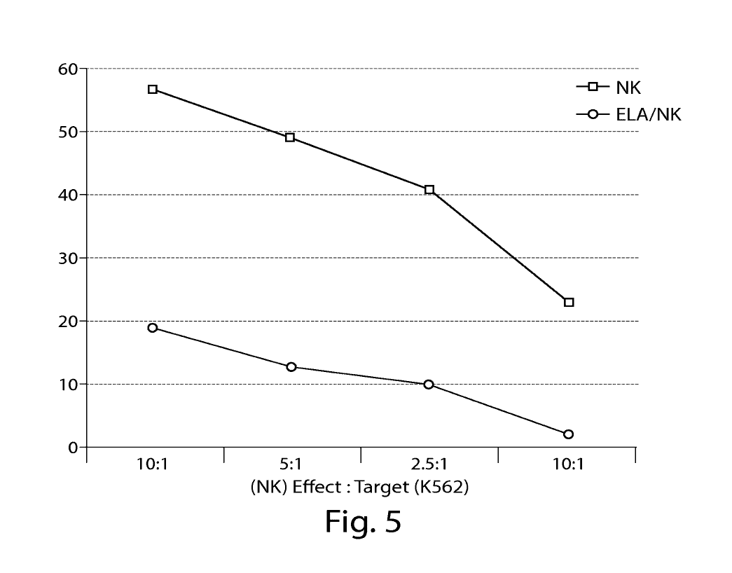

To determine whether ELA stem cells actively suppressed the innate arm of the immune response, ELA cells were co-cultured with NK cells, and the NK cells capacity to kill its target cell, K562, was assessed. NK cells were cultured in the absence or presence of ELA cells for 18 hours. The cultured NK cells were transferred into vessels containing the target cell and the level of cell killing was assessed by flow cytometry. This example demonstrated that the greatest level of efficiency of either group was at a 10 to 1 concentration of NK cells to K562 cells (See FIG. 5). In addition, it was observed that NK cells that were co-cultured with ELA cells prior to being presented to the target cells were less efficient at killing the target cells than NK cells cultured alone. These findings show that ELA cells efficiently suppressed the NK cell function in a dose dependent fashion.

Accordingly, the ability of an ELA stem cell population to inhibit NK cell activation and killing further confirms their ability to engraft as a tissue transplant without inducing cell mediated immune responses.

### Example 6

### Transcription of Tissue-Specific Genes by Transplants Cultured in Inducing Media

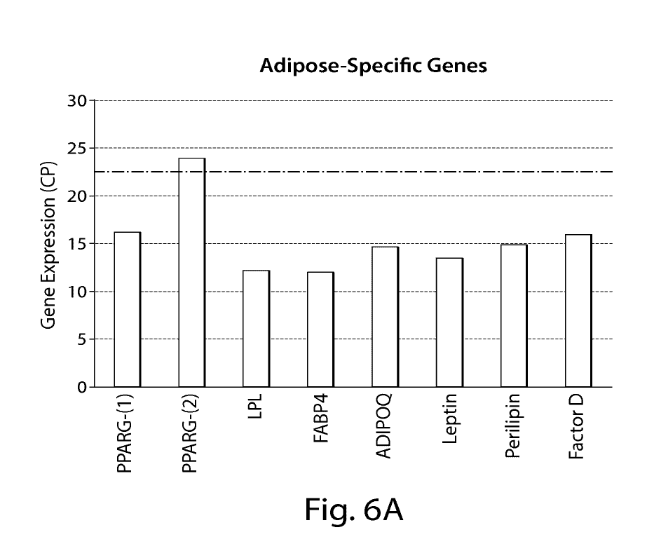

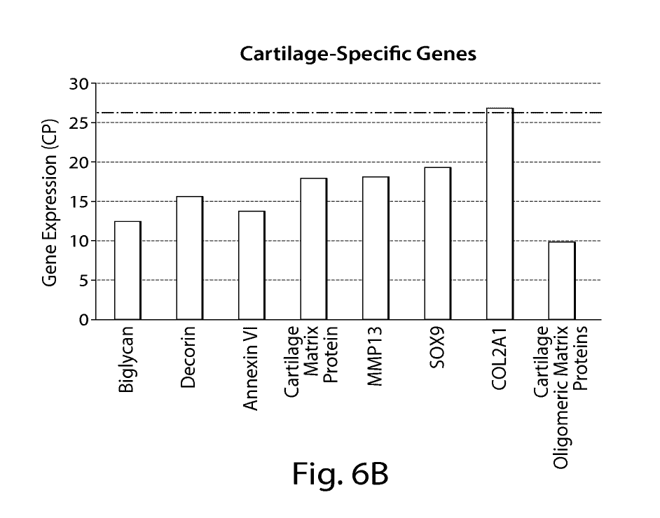

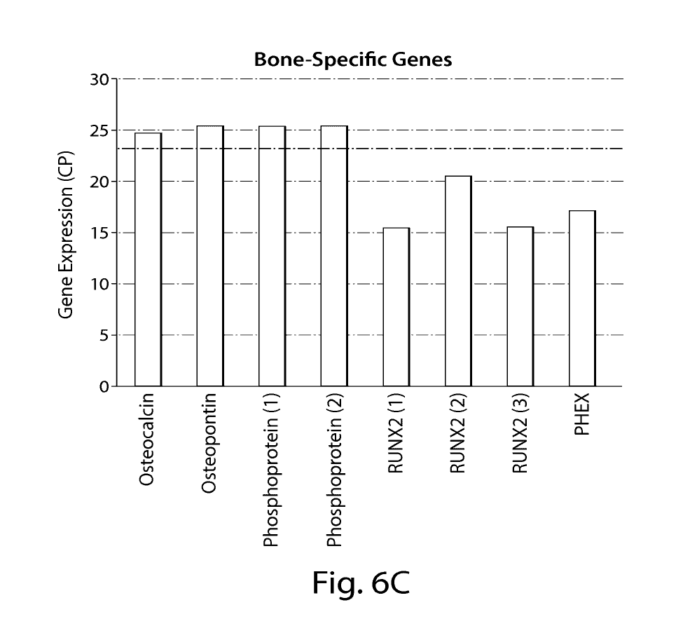

Culturing ELA cells for an adipose transplant in medium to induce adipose cell differentiation was verified using a quantitative polymerase chain reaction (QPCR) analysis. FIG. 6A shows expression of adipose-specific genes PPARG-(1), PPARG-(2), LPL, FABR4, ADIPOQ, leptin, perilipin, and factor D.

The number of PCR cycles is shown on the ordinate, and the dotted line shows number of cycles used to detect housekeeping genes. Fewer cycles in the extent of the bars indicate greater amounts of transcription of tissue-specific genes.

FIG. 6B shows QPCR analysis of an ELA cell transplant cultured in the presence of a chondrogenic medium.

Further, culture of ELA cells with chondrogenic medium as shown above induced differentiation into a cartilage-like tissue. Analysis of expression of RNA specific for cartilage-specific genes biglycan, decoin, annexin VI, cartilage matrix protein, MMP13, SOX9, COL2A1, and cartilage oligomeric matrix protein was observed (FIG. 6B).

FIG. 6C shows QPCR analysis of an ELA cell transplant cultured in the presence of an osteogenic medium.

Further, culture of ELA cells in osteoinductive medium as shown above resulted in transcription of bone-specific genes osteocatin, osteopontin, phosphoproteins (1) and (2), RUNX2(1), RUNX2(2), RUNX2(3), and PHEX (FIG. 6C).

These transcription data show that ELA transplants are suitable to be manufactured for a variety of different tissues and organs.

### Example 7

### ELA Transgene Expression

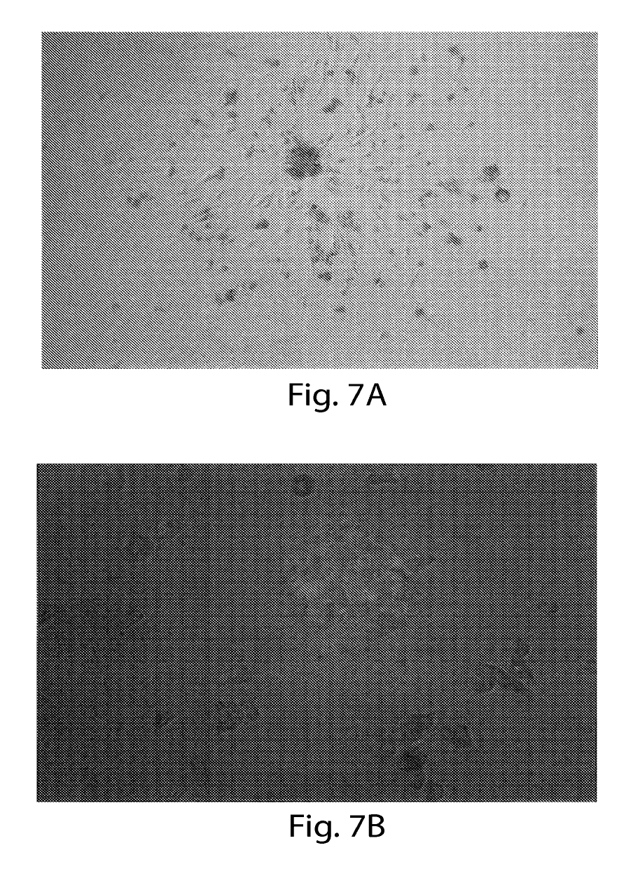

ELA transplants are useful to deliver high value proteins, as produced by cells carrying a vector encoding that gene. ELA cells were contacted with a lentivirus vector encoding green fluorescent protein (GFP) regulated by the embryonic OCT4 promoter. Cells were observed by phase contrast microscopy (FIG. 7A) and for fluorescence (FIG. 7B), and expression of the GFP was observed as shown by areas of green fluorescence visible in a color photograph, corresponding to locations of the cells. These data show that ELA cells were capable of being transformed, and expressed the GFP gene encoded by a vector from the OCT4 promoter.

### Example 8

### Comparison of ELA transplant with several commercial products for matrices

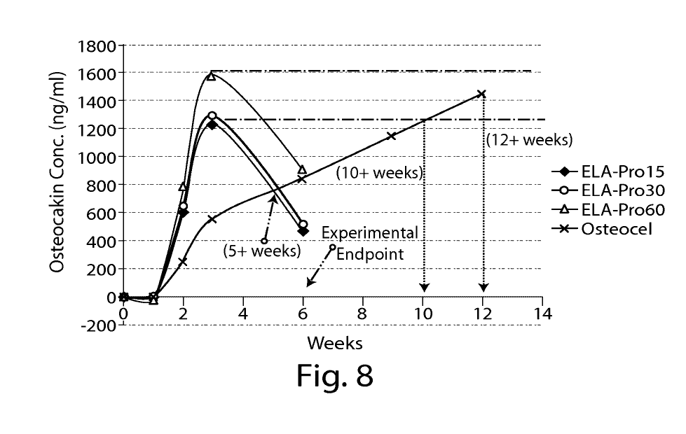

The ability of a commercially available matrix, Osteocel (Ace Surgical Supply Co., Brockton, Mass.) to differentiate into bone after 21 days in basal media was assessed. Bone growth was measured by osteocalcin concentration. No evidence of bone formation was observed in the Osteocel® after three weeks of culture. See FIG. 8. Osteocel® cultured in osteogenic media resulted in a small localized area of bone formation.

An ELA transplant of cells was added to Ostcocel® in the basal media, and bone regeneration was observed to result in more than 20 areas of bone formation. The ELA cell-Osteocel® combination cultured in the osteogenic media resulted in bone formation at faster rates. See FIG. 8, which compares Osteocel® to another matrix, ProFuse® with ELA cells seeded at three different cell counts.

In contrast to Osteocel®, matrix ProFuse® (Alphatec Spine, Inc., Carlsbad, Calif.) seeded with ELA at 15,000, 30,000, and 60,000 cell counts, resulted in substantial osteocalcin expression within 21 days.

This leads to the conclusion that the Osteocer ProFuse transplant without ELA stem cells did not efficiently generate bone with 21 days of culture. The Osteocel® scaffold does retain some osteoinductive traits, which activated osteoblastic activity when combined in a transplant with ELA cells as shown in FIG. 8, although with significantly slower kinetics than the ProFuse® matrix.

Protein and alkaline phosphatase also were measured at three and six weeks of culture in these cell matrix combinations. See Table 1. All combinations produced about the same amount of detectible protein markers at each time point. The data shown for osteocalcin in Table 1 are shown also in FIG. 8. Alkaline phosphatase (AP) produced at three and six weeks was greatest in the Osteocel® matrix, however AP is considered to be a less specific marker for osteogenic differentiation than osteocalcin. The ELA stem cells introduces cells other than stem cells into the transplant region, and the amount of AP synthesis on each matrix may be due to induction of AP protein expression in cells other than stem cells, therefore osteocalcin is a better measure of bone growth.

These data taken together show that ProFuse has greater osteoinductive properties than Osteocel®, and that for Osteocel® to promote efficient bone regeneration in osteogenic medium, ELA stem cells, such as in the ELA transplant, are added. The ELA transplant generally improves the performance of exogenous scaffolds, including cellular bone matrices, demineralized bone or synthetic and natural polymer scaffolds.

TABLE 1
Comparison of ELA cell ProFuse ® and Osteocel ®

| | ProFuse ® | ProFuse ® | ProFuse ® | |
|---|---|---|---|---|
| | 15K ELA | 30K ELA | 60K ELA | |
| Week three | cells | cells | cells | Osteocel ® |
| total protein (mg/mL) | 2 | 2 | 2.1 | 1.7 |
| Alkaline phosphatase | 9.6 | 8.8 | 8.4 | 18.4 |
| (ng/mL) | | | | |
| osteocalcin by ELISA | 1228 | 1290 | 1576 | 555 |
| (ng/mL) | | | | |
| | ProFuse ® | ProFuse ® | ProFuse ® | |
| | 15K ELA | 30K ELA | 60K ELA | |
| Week six | cells | cells | cells | Osteocel |
| total protein (mg/mL) | 1.7 | 1.8 | 2.2 | 1.8 |
| Alkaline phosphatase | 8.8 | 8.8 | 10.6 | 125.9 |
| (ng/mL) | | | | |
| osteocalcin by ELISA | 465 | 523 | 912 | 842 |
| (ng/mL) | | | | |

Further early onset of osteoblastic activity in the ELA-ProFuse combination was observed in comparison to Osteocel®. Malaval et al. 1994 J. Cell Physiol 158(3):555-572 has shown using a pure population of bone forming MSCs that the amount of osteocalcin biomarker observed reaches a maximum at 21 days and then begins to decrease. The ELA-ProFuse® transplant herein similarly produced osteocalcin biomarker in an amount that peaked at 21 days. In contrast to that observed with Osteocel® during a five-week time course, it was here observed that osteoblastic activity was significantly delayed compared to the ELA15k-ProFuse and ELA30kProFuse® levels, and osteoblastic activity resulting from Osteocel® remained substantially less than that the ELA60k-ProFuse level of bone formation.

These data show that the ELA-ProFuse transplants induce bone formation at an earlier time point than ^{Osteocel}® and achieve a greater level of bone formation overall. The data extrapolated to 14 weeks show that it would take 1cc of Osteocel® more than 10 weeks to achieve a level of osteoblastic activity comparable in amount to ELA15k-ProFuse®, and 14 weeks to achieve the amount comparable to ELA60kProFuse® at 21 days.

### Example 9

### Osteoinductive Function of ELA Cells In Vivo

ELA cells were combined with each of scaffolds ProFuse and ground bone matrix (125-850 microns gauge) to formulate transplants into subjects (rats) and were visualized by fluoroscopic assessment (X-ray) at each of the following time points: two weeks, four weeks and six weeks after implantation. After six weeks, rats were sacrificed and tissue was removed for staining with hematoxylin and eosin (H&E) and for human class I MHC. Controls and comparisons included groups of transplants prepared with each of the two scaffold materials, and with bone marrow aspirate and MSC cells. A semi-quantitative scoring method rated bone at the epiphyseal region adjacent to the implantation site as follows: grade 0 if no calcification/mineralization was evident; grade 1 if calcification/mineralization was present and hypodense compared to bone; grade 2 if calcification/mineralization was present and isodense to bone; and grade 3 if calcification/mineralization was present and hyperdense.

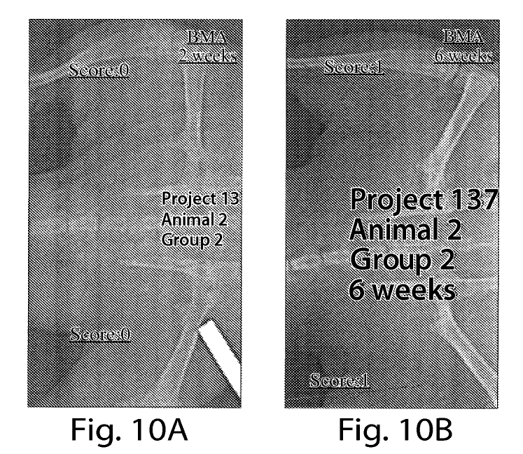

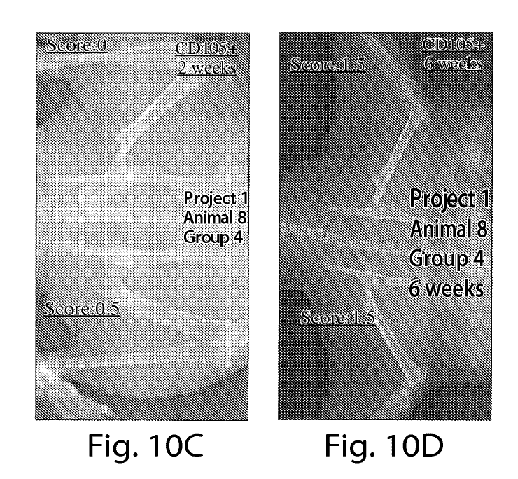

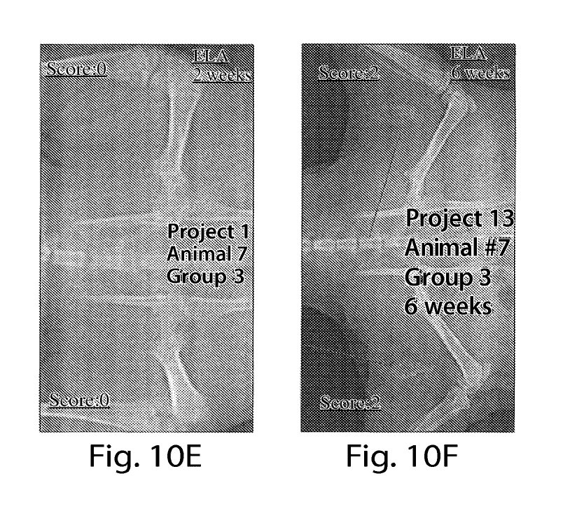

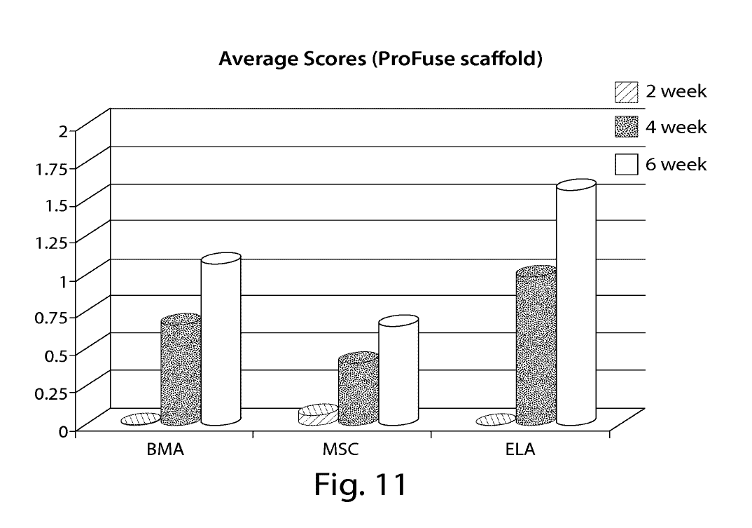

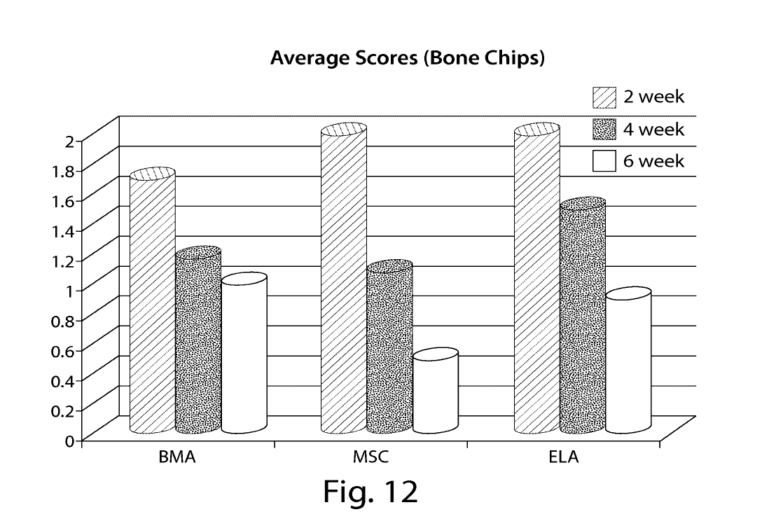

Fluoroscopic data from controls showed that at week two, human bone marrow aspirate with ProFuse yielded a score of 0, and a score of 1 at six weeks (FIGS. 10A and 10B); with CD105 MSC and ProFuse, a score of 0.5 at two weeks and 1.5 at six weeks (FIGS. 10C and 10D). In comparison, ELA cells with ProFuse yielded a score of 0 at two weeks, and a score of 2 at six weeks (FIGS. 10E and 10F). Thus, the ELA/ProFuse transplant was found to result in 46% better density than transplants using this matrix and bone marrow aspirate, and 136% better than MSC. See FIG. 11. ELA also yielded better scores using bone chip scaffold than did bone marrow aspirate and MSC at each time point. FIG. 12. Further, 100% of the sites of implantation were visible for each of the Profuse with ELA cells and bone marrow aspirate, but only 50% of with MSC, at the six week time point. Using the bone chip scaffold, 100% of the implantation sites were visible at six weeks for ELA, but only 33% of sites with bone marrow aspirate or with MSC cells. See FIG. 12.

Histological data showed evidence of new bone formation in all three types of ProFuse transplants, ELA cells, bone marrow aspirate and MSC cells. The evidence included synthesis of osteocalcin, osteopontin, and APase, and the MHC class I analysis revealed the presence of human cells in rat recipients receiving these transplants. In contrast, no evidence of new bone formation was seen for bone chip scaffold in any of the transplants, and non-specific staining was observed for each of the protein analyses. The presence of human cells was detected, including presence of multinucleate cells, indicating induction of phagocytic reaction to necrotic bone.

These data show in vivo success of an ELA transplant using ProFuse for repair of bone defects. Further, data show that ELA transplants result in more reproducible bone formation than MSC cells.

### Example 10

### Biocompatibility of ELA Cells In Vivo

ELA cells in a cryopreservation solution with dimethylsulfoxide (DMSO) and a control of ELA cells in sterile saline, were dripped evenly over at least two levels of surgically exposed lumbar vertebrae (L3/L4/L5) of subject rats. Controls were performed using the DMSO cryopreservation solution without cells, and saline without cells (sham treatments) Animals were monitored by daily manual palpation and by radiography for four weeks, and then tissue was subjected to histopathological and hematological evaluation.

The results showed no changes in the experimental recipient animals compared to the sham treatment controls, indicating that the ELA cells did not cause any pathological response in the recipients.

### Example 11

### A Spinal Allograft Composition and Method

AlphaGRAFT® ProFuse® is a demineralized human bone scaffold produced by Alphatec Spine (Carlsbad, Calif.). AlphaGRAFT® is designed to provide an environment for bony ingrowth. The reading of the scaffold with a transplant of ELA stem cells results in the osteogenic environment of the scaffold inducing the ELA stem cells to differentiate into osteoprogenitor cells, e.g., osteoblasts, and to effectuate rapid colonization of the scaffold with resultant bone growth. This result has been demonstrated in vitro, using basal media to support cell metabolism. Likewise, bone growth was observed in vivo, as a xenotransplant in a rodent intramuscular study. With the xenotransplant, no substantially aberrant immune response was seen, and the xenotransplant was not rejected by the mammalian host immune system.

In a human patient in need of vertebrate fusion, a surgeon isolates the appropriate vertebrae and incorporates the AlphaGRAFT® ProFuse® scaffold as directed by the manufacturer. The surgical team is provided with a cryogenically preserved transplant, comprising a sterile, isotonic, buffered solution having a population of about thirty to about seventy-five thousand ELA® brand osteoprogenitor cells (i.e., ELA stem cells, from Parcell Laboratories, Newton, Mass.), the cell dosage depending on the size and location of the vertebrae to be fused. The transplant is allogeneic to the recipient patient, and has not been matched according to tissue matching protocols. The transplant is held on dry ice, then the cells are thawed in a sterile 37° C. water bath when the surgeon is ready to initiate the transplant. The transplant is performed by seeding the scaffold with the transplant. The surgeon concludes the procedure and monitors the recipient patient for surgical recovery, allograft rejection and bone growth/fusion of the affected vertebrae.

Similar procedures are suitable for cervical fusion, in which case the transplant incorporates fewer ELA stem cells, e.g., preferably about 10,000 to about 50,000.

### Example 12

### ELA Transplants and Reducing Incidence of Pseudoarthrosis

The term “pseudoarthrosis” means false joint. A surgeon uses this term to describe either a fractured bone that has not healed or an attempted fusion that has not been successful. Pseudoarthrosis generally describes motion between two bones that should be healed or fused together. There is usually continued pain when the vertebrae involved in a surgical fusion do not heal. The pain may increase over time. The spinal motion can also stress the metal hardware used to hold the fusion, possibly causing breakage. The patient may need additional surgery for a pseudoarthrosis condition.

It is here envisioned that the more time that elapses prior to the onset of healing in the spine, the greater the probability of an incidence of pseudoarthrosis. The addition of ELA cells to site of injury will hasten the onset of bone regeneration, as shown in FIG. 8, thereby decreasing the incidence of pseudoarthrosis.

### Example 13

### Differentiation of ELA Cells in a Putty Matrix

ELA cells were seeded into a matrix commercially available from Etex Corp. Cambridge, Mass. Matrices CarriGen and CarriCell are each available as a liquid putty that solidifies at 37 C. ELA cells were seeded on blocks of the CarriCell putty and were incubated in individual wells of a multi-well culture plate under conditions suitable for development of osteocytes, and synthesis of the developmental marker alkaline phosphatase was analyzed by stain.

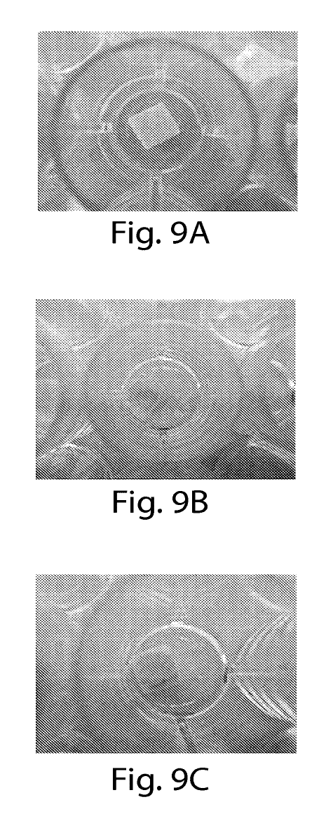

Data shown in FIG. 9 indicate that ELA cells differentiated into osteocytes. FIG. 9A is a photograph that shows a block with cells prior to staining, and FIGS. 9B and 9C show stained cells. These results indicate that a transplant of ELA stem cells is capable of inducing measuring levels of bone synthesis on the Etex matrix. Similar results were obtained using the Helos® matrix commercially available from Johnson & Johnson.

### Example 14

### Differentiation of ELA Cells in a Chondrogenic Environment

Seeding chondrogenic cells in alginate beads, Masuda et al. U.S. Pat. No. 6,197,061, showed that the cells elaborate a cartilage specific cell associated matrix having proteins characteristic of cartilage, and upon further culture generated a flexible cartilage like material suitable for press fitting into a cartilage defect. Further, Pfister et al. PCT/US03/14996 2003 showed that these cells removed from the beads and seeded on a bone substitute scaffold infiltrated pores in the scaffold and generated a cartilage with a tissue specific polarity for inserting into a bone defect, so that following transplant, cartilage tissue develops on one surface of the scaffold, and another opposite surface remains suitable for growth of bone cells into pores of that surface.

It is here envisioned that ELA cells seeded in alginate beads similarly differentiate into a cartilage-like tissue, and elaborate the cartilage specific cell associated matrix. ELA cells are further cultured, the alginate is removed according to Masuda et al. and Pfister et al., and the ELA cells are used to generate the flexible cartilage suitable for press fitting, or are further cultured on a bone substitute material to generate a transplant suitable for insertion into a cartilage defect. Specifically envisioned is a scaffold having tissue specific polarity, that can repair a bone and soft tissue defect, for example an ACL, transplant, or any application requiring joining connective tissue to bone tissue.

### Example 15

### Dermal, Epithelial and Endothelial Transplants

The ELA brand stem cell allograft is suitable for dermatological grafting procedures, as it can differentiate into hypodermis, dermis and epidermis, as well as the various glandular, connective, nervous and circulatory tissues in the various cutaneous layers. The transplant has wide application in the treatment of, for example burn victims, trauma victims, as well as in the treatment of diabetic ulcers, skin grafts, and even cosmetic procedures, due to the pluripotent properties of the ELA stem cells.

As described above, the ELA stem cell transplant may be induced toward specific cellular lineages, and may be incorporated into various natural or artificial matrices that support cellular ingrowth and may further provide a cell lineage-inducing environment. While the choice of matrix depends on the particular application, as does the required ELA transplant size (e.g., ELA cell dosage), the following describes non-limiting exemplary uses of ELA transplants. While allografts are described, the invention and the applications that follow include the use of syngeneic ELA transplants as well.

Regrowth of cutaneous tissue may further include a need to repair connective tissues, basement membrane tissues, underlying musculature and nerves, etc. Conventional skin grafts are designed to regrow skin, and will not repair the entirety of a wound site. Accordingly, a pluripotent graft is desirable. Functions of epithelial cells include secretion, selective absorption, protection, transcellular transport and detection of sensation. As a result, they commonly present extensive apical-basolateral polarity (e.g. different membrane proteins expressed) and specialization. The ELA brand transplants desirably provide for growth of cells having such polar orientations.

Skin substitutes provide a surrogate for skin functions. Preferably skin substitutes incorporate themselves in to the repaired wound, and more preferably induce healing. Common skin substitutes include allografts, xenografts, cultured skin grafts, acellular dermal allografts and artificial biosynthetic membranes.

Skin allografts and xenografts retain the donor cells and are biologic dressings only, and are ultimately rejected by the recipient's immune system, necessitating removal prior to definitive wound treatment or skin grafting. Permacol (Tissue Science Laboratories, Hampshire, UK) is an exemplary xenograft tissue that has been treated to extend the lifespan and microbial resistance of the graft. EZ-Derm® (Brennen Medical) is composed of cross-linked porcine collagen and serves more as a scaffold than a dressing, but is a xenograft.

An ELA stem cell transplant is used to augment skin allografts or xenografts. Currently preferred treatments utilize a transplant size that deposits approximately 1000 to 10,000 cells per sq cm of treatment area, although 10-1,000,000 per sq cm of cells may be utilized depending on the complexity of the defect (e.g., types of tissues involved in the repair, wound depth, and location of the treatment site). A surgeon prepares the transplant site according to current medical practices, applies the ELA transplant to the site, and utilizes the skin allograft or xenograft as a biologic dressing while the ELA transplant restores the treated tissues under the dressing. Since the dressing is itself immunogenic, a larger ELA transplant is preferred, due to its immunomodulatory properties, thereby permitting longer duration of the dressed wound.

A patient's own epithelial cells may be harvested and grown in culture for use as a larger epidermal autograft. These autografts address the epidermal layer only and are typically quite thin. Cultured epidermal autograft (CEA), such as Epicel® (Genzyme Biosurgery, Cambridge, Mass) and Laserskin® (Fidia Advanced Biopolymers, Abano Terme, Italy) use a biopsy from the patient that is expanded via culture techniques in the laboratory setting to produce a sheet of autogenous keratinocytes for grafting. Cultured autografts can provide for treatment of large surface area defects using a small amount of donor tissue, but this type of graft has been associated with high rates of infection and graft loss, confirming the importance of the dermal layer in skin grafting. Cultured skin substitute (CSS) are an attempt to enhance graft performance.

An ELA stem cell transplant is used to augment autografts and cultured skin substitutes. Currently preferred treatments utilize a transplant size that deposits approximately 1000 to 10,000 cells per sq cm of treatment area, although 10-1,000,000 per sq. cm of cells may be utilized depending on the complexity of the defect (e.g., types of tissues involved in the repair, wound depth, and location of the treatment site). A surgeon prepares the transplant site according to current medical practices, applies the ELA transplant to the site, and utilizes the cultured skin in conjunction with the ELA transplant. Lower doses of ELA cells can be used if the cultured skin is abundant, and the ELA stem cells and included immune cells in the ELA transplant advantageously provide a measure of protection against microbial attack on the grafted cells.

Acellular dermal allografts, such as AlloDerm (LifeCell, Branchburg, NJ), are cadaveric dermis grafts that serve as a scaffold. AlloDerm® has been used for repair of skin defects, but has also been used for abdominal wall reconstruction, coverage of implantable prostheses, single-stage soft tissue defect reconstruction, and for the repair of head and neck defects. Other acellular dermal allografts include Strattice® (LifeCell, Branchburg, NJ), SurgiMend® (TEI Biosciences, Boston, Mass.), GraftJacket® (Wright Medical Technologies, Inc, Arlington, Tenn.), NeoForm® (Mentor Corporation, Santa Barbara, Calif.), and DermaMatrix® (Synthes, Inc, West Chester, Penn), which have been studied for applications such as lower extremity, craniofacial, and breast reconstruction. The Repliform® Tissue Regeneration Matrix (Boston Scientific Corporation) is an acellular human dermal allograft that functions as a matrix for fibroblast ingrowth. The hMatrix™ (Bacterin International Holdings) is an acellular matrix made from donated human dermal tissue. FlexHD® Acellular Hydrated Dermis (Ethicon) is an acellular dermal matrix derived from donated human allograft skin. FlexHD has been demonstrated to be effective for use in the repair of abdominal wall defects, full-thickness burns, and breast reconstruction post-mastectomy, and can be used to cover and reinforce damaged or inadequate integumental, connective, and soft tissues. The INTEGRA® Dermal Regeneration Template (Integra LifeSciences Corporation) is a bilayer matrix that provides a scaffold for dermal regeneration. It is a bilaminate membrane consisting of a porous collagen layer (dermal analogue) bonded to a thin silicone layer (temporary epidermis). The dermal layer becomes revascularized and populated by cells from the patient's own underlying tissue over 7-21 days. Once this process is complete, an ultrathin split-thickness skin graft, or epidermal autograft, is placed over the new dermis after removal of the silicone layer from the new dermal layer. The Puros Dermis Allograft Tissue Matrix (Zimmer Dental) is an acellular de-antigenated allograft dermal tissue for dental applications, particularly gum and periodontal repair, further illustrating the myriad uses of dermal grafts to treat defects of tissues other than cutaneous tissue. GammaGraft® (Promethean Life Sciences) is a gamma-irradiated cadaveric allograft, that contains both epidermal and dermal components.

An ELA stem cell transplant is used to augment acellular dermal allografts. Currently preferred treatments utilize a transplant size that deposits approximately 1000 to 10,000 cells per sq cm of treatment area, although 10-1,000,000 per sq. cm of cells may be utilized depending on the complexity of the defect (e.g., types of tissues involved in the repair, wound depth, and location of the treatment site). A surgeon prepares the transplant site according to current medical practices, applies the ELA transplant to the site, and utilizes the acellular dermal allograft in conjunction with the ELA transplant. The ELA stem cells in the transplant exhibit in-growth into the acellular scaffold, colonizing it and repairing the wound tissue. Certain of the above acellular scaffolds are meant to be removed in part or in whole after a time, in which case the procedure is modified to treat such scaffolds as partial or complete dressings. However, most such acellular scaffolds are intended to be left in place.

Biobrane® (UDL Laboratories, Inc., Rockford, Ill.) is a biosynthetic dressing composed of a silicone membrane (the epidermal layer) coated on one side with porcine collagen and imbedded with nylon mesh (the dermal layer). When used to cover partial-thickness wounds, the mesh adheres to the wound until healing occurs below. Biobrane® is removed from any full-thickness wound prior to skin grafting. Biobrane® provides a biosynthetic dressing for burn wounds, particularly in the pediatric population, but has applications in patients with TEN, chronic wounds, and following skin resurfacing. Cellular dermal allografts are typically composed of a collagen or polymer-based scaffold that is seeded with fibroblasts from a donor cadaver. These products, including ICX-SKN® (Intercytex Ltd, Manchester, UK), TransCyte®, and Dermagraft®, have reported use in coverage of partial- and full-thickness wounds. TransCyte (Advanced Tissue Sciences, Inc., La Jolla, Calif) is a nylon mesh incubated with human fibroblasts that provides a partial dermal matrix with an outer silicone layer as a temporary epidermis. It is indicated for use in deep partial or excised full-thickness wounds prior to autogenous skin graft placement. It is removed or excised prior to grafting full-thickness wounds. Dermagraft® (Advanced Tissue Sciences, Inc., La Jolla, Calif.) consists of human neonatal fibroblasts cultured on Biobrane. The neonatal fibroblasts are seeded into the nylon mesh. Approximately two weeks after application, the silicone membrane is removed and the wound bed grafted with a split-thickness skin graft. Dermagraft is a dressing and does not provide full dermal scaffolding, thus requiring standard depth split-thickness skin grafts.

An ELA stem cell transplant is used to augment biosynthetic dressings. Currently preferred treatments utilize a transplant size that deposits approximately 1000 to 10,000 cells per sq cm of treatment area, although 10-1,000,000 per sq. cm of cells may be utilized depending on the complexity of the defect (e.g., types of tissues involved in the repair, wound depth, and location of the treatment site). A surgeon prepares the transplant site according to current medical practices, applies the ELA transplant to the site, and utilizes the biosynthetic dressing in conjunction with the ELA transplant. The ELA stem cells attenuate host immune responses against the seeded fibroblasts, and function as described above for dressings.

Composite allografts are bilayer products, exemplified by Apligraf® Organogenesis, Inc., Canton, Mass.), having a dermal component of bovine collagen and incorporating neonatal fibroblasts combined with an epidermal layer formed by neonatal keratinocytes, and Orcel® (Ortec International, Inc., New York, N.Y.), a bovine collagen sponge coated with neonatal allogeneic keratinocytes. As allografts, however, they cannot be used as permanent skin substitutes, as they will be rejected eventually by the patient's immune system. These materials have primarily been used in the treatment of chronic wounds and donation sites, and as an overlay dressing on split-thickness skin grafts.

An ELA stem cell transplant is used to augment composite allografts. Currently preferred treatments utilize a transplant size that deposits approximately 1000 to 10,000 cells per sq cm of treatment area, although 10-1,000,000 per sq. cm of cells may be utilized depending on the complexity of the defect (e.g., types of tissues involved in the repair, wound depth, and location of the treatment site). A surgeon prepares the transplant site according to current medical practices, applies the ELA transplant to the site, and utilizes the composite allograft in conjunction with the ELA transplant. As ELA stem cells are pluripotential, they are capable of differentiating into multiple cell types and even exhibit polarity in specific cell layers.

The above ELA allografts may further be used in combination with composite allografts, biosynthetic dressings, acellular dermal allografts, autografts and cultured skin substitutes, skin allografts and xenografts, in conjunction with negative pressure wound therapy (see, for example the wound treatment systems of U.S. Pat. Nos. 7,534,240 and 7,361,184 and dressings/gels described in U.S. Pat. Nos. 7,005,556 and 6,379,702, all hereby incorporated herein by reference). Grafting wound healing factors such as the ELA transplant into a wound prior to application of a dressing or allograft etc., and/or a porous pad of a negative pressure system provides such a method for treatment. For example, in a diabetic ulcer, the wound is debrided and the ELA transplant is administered by one or more injections around the periphery of the ulcer site.

Cell number ranges for ELA transplants in negative pressure applications reflect those given above for various graft types. Following transplant, the porous pad which is permeable to fluids and adapted for positioning within a sealable space defined in part by a wound surface is obtained; and a tube is inserted through the pad, the tube having a first end in fluid communication with the porous pad and a second end in fluid communication with a vacuum source. The vacuum source is adapted to apply negative pressure to the porous pad through the tube. The ELA transplant may be first contacted with cellular growth and differentiation factors prior to transplant. Accordingly, an ELA transplant is used to repair hypodermal, dermal and epidermal tissues of skin, as well as connective, sebaceous, vascular endothelial, cardiac muscle and neural tissues at a wound site, arising, e.g., from burns and/or trauma. In particular, the accelerated tissue healing properties of negative pressure treatment serve to induce robust tissue differentiation capabilities in such ELA transplants, while inducing and maintaining immune response attenuation with respect to the transplant but enhanced immune detection to pathogenic microorganisms and response to infection.

### REFERENCES

1. Stern R. McPherson M, Longaker, Mont. Histologic study of artificial skin used in the treatment of full thickness thermal injury. J Burn Rehabil. 1990; 11:7-13
2. IIeimbach D, Luteman A, Burke JF, et al. Artificial dermis for major burns: a multi-center randomized clinical trial. Ann Surg 1988; S208:313-320
3. Data on file, Integra LifeSciences Corporation
4. Michaeli D, McPherson M. Immunologic study of artificial skin used in the treatment of thermal injuries. J Burn Care Rehabil. 1990; 11:21-26.
Petrungaro P. Correction of Iatrogenic Gingival Recession in the Esthetic Zone. Inside Dentistry. 2007; 11:2-4.
Schoepf C. Allograft Safety: Efficacy of the Tutoplast Process. Implants: Int J Oral Implantol. 2006; 7:10-15.
Onur R. Singla A. Solvent-dehydrated cadaveric dermis: a new allograft for pubovaginal sling surgery. J. Urol. 2005; 12:801-805.
Patino M. Neiders M. Andreana S. Noble B. Cohen R. Collagen: An Overview. Implant Dent. 2002; 11:280-285.

### INCORPORATION BY REFERENCE

The entire contents of all patents published patent applications and other references cited herein are hereby expressly incorporated herein in their entireties by reference.

### EQUIVALENTS

Those skilled in the art will recognize, or be able to ascertain using no more than routine experimentation, numerous equivalents to the specific procedures described herein. Such equivalents are considered to be within the scope of this invention and are covered by the following claims.

What is claimed is:

1. A transplant composition comprising: a T-cell- or NK-cell-suppressive amount of a non-expanded population of human early lineage adult (ELA) stem cells, an immune cell population comprising plasmacytoid dendritic cells, and a cryopreservative, wherein the human ELA stem cells express at least one of Oct4, Nanog and Sox2, and do not detectably express CD13, CD45, CD90 or CD34, and wherein the transplant composition is without detectable erythrocyte cells.

2. The transplant composition of claim 1, wherein the transplant is an allograft.

3. The transplant composition of claim 2, wherein the allograft is not MHC matched to the human transplant recipient.

4. The transplant composition of claim 1, wherein the transplant includes at least one component selected from the group consisting of plasma, cell culture medium, an antibacterial agent, a growth factor, a vitamin, and a hormone.

5. The transplant composition of claim 1, further comprising a carrier having a matrix, wherein the matrix conforms substantially to its insertion site and provides a structurally stable, three dimensional surface that retains the transplant and supports ingrowth of ELA stem cells into the matrix at the insertion site.

6. The transplant composition of claim 1, wherein the transplant promotes the formation of a tissue selected from the group consisting of: connective tissue; bone; and dermal tissue.

7. The transplant composition of claim 1, wherein the human ELA stem cell numbers in the transplant comprise about 5000 to about 5×10⁶ human ELA stem cells.

8. The transplant composition of claim 1, wherein the transplant composition is cryogenically frozen.
